(19) United States
(12) Patent Application Publication
(10) Pub. No.: US 2012/0142111 A1
(43) Pub. Date: Jun. 7, 2012

(54) NANOMATERIAL-CONTAINING SIGNALING COMPOSITIONS FOR ASSAY OF FLOWING LIQUID STREAMS AND GEOLOGICAL FORMATIONS AND METHODS FOR USE THEREOF

(76) Inventors: James M. Tour, Bellaire, TX (US); Dmitry Kosynkin, Houston, TX (US); Michael Wong, Houston, TX (US); Mason Tomson, Houston, TX (US); Jacob Berlin, Houston, TX (US); Ashley Leonard, Houston, TX (US); Jay Lomeda, Houston, TX (US); Wei Lu, Houston, TX (US); Jie yu, Katy, TX (US); Lunliang Zhang, Beijing (CN); Amy Kan, Houston, TX (US)

(21) Appl. No.: 13/378,440

(22) PCT Filed: Jun. 11, 2010
(86) PCT No.: PCT/US10/38363

§ 371 Date: Feb. 23, 2012

Related U.S. Application Data
(60) Provisional application No. 61/187,071, filed on Jun. 15, 2009.

(51) Int. Cl. G01N 21/64; C07D 303/32; C07D 263/52; G01N 33/00; C09K 11/06; G01N 21/29; C07C 63/33; B82Y 15/00

(52) U.S. Cl. 436/27; 436/56; 436/164; 549/543; 562/405; 548/217; 252/408.1; 252/301.16; 428/402; 73/53.01; 977/773

(57) ABSTRACT

Compositions containing a transporter component and a signaling component and a method for using said compositions for analyzing porous media and flowing liquid streams, specifically for measuring pressure, temperature, relative abundance of water, pH, redox potential and electrolyte concentration. Analytes may include petroleum or other hydrophobic media, sulfur-containing compounds. The transporter component includes an amphiphilic nanomatenal and a plurality of solubilizing groups covalently bonded to the transporter component. The signaling component includes a plurality of reporter molecules associated with the transporter component. Said reporter molecules may be releasable from the transporter component upon exposure to at least one analyte. The reporter molecules may be non-covalently associated with the transporter component, or the reporter molecules are covalently bonded to the transporter component. Furthermore, said compositions and methods may be used to actively enhance oil recovery and for remediation of pollutants.

### CROSS-REFERENCE TO RELATED APPLICATIONS

This application claims priority to U.S. Provisional Patent Application 61/187,971, filed Jun. 15, 2009, which is incorporated by reference in its entirety herein.

### STATEMENT REGARDING FEDERALLY SPONSORED RESEARCH

This invention was made with Government support under grant number DE-FC-36-05GO15073 awarded by the United States Department of Energy, grant number W81XWH-08-2-0143 awarded by the United States Army, grant number 09-S568-064-01-C1 awarded by Universal Technology Corporation via pass-through funding from the Air Force Research Laboratory grant number FA8650-05-D-5807, and grant number EEC-0647452 awarded by the National Science Foundation. The invention was also made with Government support under grant number FA8650-07-2-5061 awarded by the Air Force Office of Scientific Research under the Air Force Research Laboratory Consortium for Nanomaterials for Aerospace Commerce and Technology (CONTACT). The Government has certain rights in the invention.

### BACKGROUND

Due to the rapid increase of worldwide oil demand, improved methods for petroleum production are becoming more crucial. Petroleum production can be enhanced if 1) the geology of oilfields can be accurately mapped and 2) the flow from injection wells to production wells can be characterized. Along these lines, tracers have been used for numerous oilfield applications. Although tracers are efficient in conveying an entry-exit correlation, they provide little if any information on the environment existing between the entry and exit locations.

Nanotechnology has been proposed for many oilfield applications such as, for example, enhanced oil recovery, and drilling and scale control, but there have yet to be any uses disclosed for using nanotechnology in mapping and quantifying various parameters of oilfields. The lack of research in this regard is surprising given the beneficial electrical, optical and magnetic properties of many nanomaterials. Although there is a tremendous potential upside for using nanomaterials in oilfield applications, there remain a number of challenges to be overcome for implementing their use in this field. These challenges include, for example, complicated local conditions such as high salinity, low permeability, and heterogeneous rock properties. The presence of petroleum and/or natural gas intermixed with an aqueous environment further complicates analysis of the local geological conditions present in an oilfield.

In view of the foregoing, more efficient methods for assaying local conditions in a geological structure, such as, for example, a petroleum reservoir, are desperately needed in the art to sense and record local environmental conditions. Nanomaterial-based assay methods present a unique opportunity to study local environmental conditions such as, for example, relative quantities of oil and water, salinity, pH, redox potential, pressure, temperature and presence of sulfur-containing compounds, through eliciting a chemical or structural change in the presence of an analyte. In addition, nanomaterial-based assays allow more than one local environmental condition to be assayed at a time. Nanomaterial-based assay methods represent a potentially significant advance over tracer technology currently utilized in the art. In general, the compositions and methods disclosed herein accomplish the aforesaid goals. In addition, through simple extension of the presently disclosed compositions and methodologies, any flowing liquid stream such as, for example, a natural stream or a wastewater stream can be efficiently assayed. Furthermore, the compositions and methodologies can be simply extended to actively enhance oil recovery or remediate contamination from various sources.

### SUMMARY

In various embodiments, compositions containing a transporter component and a signaling component are described herein. In some embodiments, the transporter component includes an amphiphilic nanomaterial and a plurality of solubilizing groups covalently bonded to the amphiphilic nanomaterial, and the signaling component includes a plurality of reporter molecules associated with the transporter component. At least a portion of the plurality of reporter molecules is releasable from the transporter component upon exposure to at least one analyte of interest.

In some embodiments of the compositions, the transporter component includes an amphiphilic nanomaterial and a plurality of solubilizing groups covalently bonded to the amphiphilic nanomaterial, and the signaling component includes a first plurality of reporter molecules covalently bonded to the transporter component. At least a portion of the first plurality of reporter molecules is cleavable from the transporter component upon exposure to at least one analyte of interest. In further embodiments, the compositions further include a second plurality of reporter molecules that are either covalently bonded to the transporter component or not covalently bonded to the transporter component. In such embodiments, the first plurality of reporter molecules and the second plurality of reporter molecules are operable to detect different analytes of interest.

In other various embodiments, compositions of the present disclosure include a transporter component that is operable to become covalently bonded to a reporter molecule upon exposure to at least one analyte of interest. The transporter component includes an amphiphilic nanomaterial and a plurality of solubilizing groups covalently bonded to the amphiphilic nanomaterial.

In still other various embodiments, compositions of the present disclosure include a transporter component having an amphiphilic nanomaterial that is collapsible at a predetermined pressure and a plurality of solubilizing groups covalently bonded to the amphiphilic nanomaterial.

In other various embodiments, assay methods utilizing the present compositions are described herein. In some embodiments, the methods include providing a composition having a transporter component and a signaling component, exposing the composition to a liquid medium containing at least one analyte of interest and recovering the composition from the liquid medium after exposing. The transporter component includes an amphiphilic nanomaterial and a plurality of solubilizing groups covalently bonded to the amphiphilic nanomaterial. The signaling component includes a plurality of reporter molecules non-covalently associated with the transporter component. At least a portion of the plurality of reporter molecules is released from the transporter component upon exposure to the at least one analyte of interest. The plurality of reporter molecules is present in a first concentration in the composition prior to exposing and in a second concentration after exposing. The methods further include assaying the composition to determine the second concentration. In further embodiments, the methods also include assaying the liquid medium for the portion of the plurality of reporter molecules released from the transporter component.

In other various embodiments, methods of the present disclosure include providing a composition having a transporter component and a signaling component, exposing the composition to a liquid medium containing at least one analyte of interest and recovering the composition from the liquid medium after exposing. The transporter component includes an amphiphilic nanomaterial and a plurality of solubilizing groups covalently bonded to the amphiphilic nanomaterial. The signaling component includes a plurality of reporter molecules covalently bonded to the transporter component. At least a portion of the plurality of reporter molecules is cleaved from the transporter component upon exposure to the at least one analyte of interest. The plurality of reporter molecules are present in a first concentration in the composition prior to exposing and in a second concentration after exposing. The methods further include assaying the composition to determine the second concentration. In further embodiments, the methods also include assaying the liquid medium for the portion of the plurality of reporter molecules cleaved from the transporter component.

In still other various embodiments, methods of the present disclosure include providing a composition having a transporter component containing an amphiphilic nanomaterial and a plurality of solubilizing groups covalently bonded to the amphiphilic nanomaterial, exposing the composition to a liquid medium containing at least one analyte of interest, recovering the composition from the liquid medium after exposing, and assaying the composition to determine the concentration of the at least one analyte of interest in the composition. At least a portion of the at least one analyte of interest becomes associated with the transporter component during exposing.

In still additional embodiments, methods of the present disclosure include providing a composition having a transporter component and a signaling component, injecting the composition into a geological formation containing at least one analyte of interest, and recovering the composition from the geological formation after a period of time. The transporter component includes an amphiphilic nanomaterial and a plurality of solubilizing groups covalently bonded to the amphiphilic nanomaterial. The signaling component includes a plurality of reporter molecules associated with the transporter component. At least a portion of the plurality of reporter molecules is released from the transporter component upon exposure to the at least one analyte of interest. The plurality of reporter molecules are present in a first concentration in the composition prior to injection and in a second concentration after being recovered. The methods further include assaying the composition to determine the second concentration. The ratio of the second concentration to the first concentration can be correlated with an amount of the at least one analyte of interest in the geological formation. In further embodiments, the methods further include assaying the geological formation for the portion of the plurality of reporter molecules released from the transporter component.

In still other various embodiments, methods of the present disclosure include providing a first composition containing a transporter component containing an amphiphilic nanomaterial and a plurality of solubilizing groups covalently bonded to the amphiphilic nanomaterial and a signaling component containing a first identification tag covalently bonded to the transporter component, injecting the first composition into a geological formation, providing a second composition containing a transporter component containing an amphiphilic nanomaterial and a plurality of solubilizing groups covalently bonded to the amphiphilic nanomaterial and a signaling component containing a second identification tag covalently bonded to the transporter component, injecting the second composition into the geological formation after a first period of time separating the injection of the first composition and the injection of the second composition, and assaying the geological formation for the presence of the first composition and the second composition. Concentrations of the first composition and the second composition in the geological formation are diagnostic of physical changes that occur in the geological formation over the first period of time. The concentration of the first composition and the second composition can also be characteristic of the internal structure of the geological formation. The methods can be extended to include any number of compositions containing an identification tag and any number of time periods over which assays are to be made. In some embodiments, the signaling components of the compositions further include a plurality of reporter molecules associated with the transporter component.

In still further embodiments, the methods include providing a composition containing a transporter component containing an amphiphilic nanomaterial and a plurality of solubilizing groups covalently bonded to the amphiphilic nanomaterial and a signaling component containing a plurality of reporter molecules associated with the transporter component and an identification tag covalently bonded to the transporter component, injecting the composition into a geological formation in a first location, recovering the composition from the geological formation in a second location over a period of time, and analyzing the composition recovered from the second location.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE DRAWINGS

For a more complete understanding of the present disclosure, and the advantages thereof, reference is now made to the following descriptions to be taken in conjunction with the accompanying drawings describing specific embodiments of the disclosure, wherein:

FIGS. 1A-1E present schematics showing some illustrative but non-limiting processes that the various compositions of the present disclosure may undergo upon exposure to one or more analytes or conditions of interest;

FIGS. 2A-2D show illustrative reactions for synthesizing oxidized nanomaterials that possess both hydrophilic and hydrophobic domains;

FIGS. 3A-3C show illustrative reactions for covalently bonding solubilizing groups to oxidized carbon nanotubes and other oxidized nanomaterials;

FIG. 4 shows illustrative fluorescence spectra (280 nm excitation) of PPO in isooctane, PPO sequestered in an oxidized carbon nanotube transporter component and an isooctane blank;

FIG. 5 shows illustrative fluorescence spectra (280 nm excitation) of PPO extracted into hexanes from an oxidized carbon nanotube transporter component, PPO sequestered in an oxidized carbon nanotube transporter component before and after partitioning with hexanes, and a hexanes blank;

FIGS. 6A and 6B show illustrative UV-VIS spectra of phenanthrene extracted into isooctane from an oxidized carbon nanotube transporter component and phenanthrene sequestered in an oxidized carbon nanotube transporter component before and after partitioning with isooctane;

FIGS. 7A and 7B show illustrative UV-VIS spectra of phenanthrene extracted into isooctane from an oxidized graphene nanoribbon transporter component and phenanthrene sequestered in an oxidized graphene nanoribbon transporter component before and after partitioning with isooctane;

FIG. 8 shows illustrative fluorescence spectra of PPO sequestered in a carbon nanotube transporter component as prepared, after passage through a column of clean sand and after passage through a column of isooctane-treated sand;

FIG. 9 shows illustrative elution profiles from sandstone of an oxidized carbon nanotube transporter component containing a sequestered ¹⁴C aromatic compound as analyzed radiometrically and an oxidized carbon nanotube transporter component as analyzed by UV-VIS spectroscopy;

FIG. 10 shows illustrative elution profiles from dolomite of an oxidized carbon nanotube transporter component containing a sequestered ¹⁴C aromatic compound as analyzed radiometrically and an oxidized carbon nanotube transporter component as analyzed by UV-VIS spectroscopy;

FIG. 11 shows a schematic of a representative synthetic route used to prepare an illustrative oxidized carbon nanotube transporter component containing a disulfide-bonded fluorescent dye;

FIG. 12 shows an illustrative elution profile for an oxidized carbon nanotube transporter component eluted through Lula soil;

FIG. 13 shows an illustrative elution profile from Lula soil for an oxidized carbon nanotube transporter component containing a sequestered ¹⁴C-radiolabelled aromatic compound;

FIG. 14 shows illustrative dolomite breakthrough plots for an oxidized graphene nanoribbon transporter component dissolved in deionized water and brine;

FIG. 15 shows illustrative sandstone and dolomite breakthrough plots for an oxidized carbon nanotube transporter component dissolved in brine;

FIG. 16 shows illustrative dolomite breakthrough plots of oxidized carbon nanotube transporter components in the presence of various salt solution concentrations;

FIG. 17 shows illustrative dolomite breakthrough plots of an oxidized carbon nanotube transporter component in the presence of various concentrations of divalent metal cations;

FIG. 18A shows an illustrative sandstone breakthrough plot for poly(vinyl alcohol)-functionalized oxidized carbon nanotubes dissolved in brine; FIG. 18B shows an illustrative dolomite breakthrough plot for poly(vinyl alcohol)-functionalized oxidized carbon nanotubes dissolved in brine;

FIGS. 19A and 19B show illustrative sandstone (FIG. 19A) and dolomite (FIG. 19B) breakthrough plots in brine for oxidized carbon nanotube transporter components functionalized with various molecular weight poly(vinyl alcohol) solublizing groups (2,000 and 9,000 molecular weight); and

FIG. 20 shows illustrative sandstone and dolomite breakthrough plots for poly(vinyl alcohol)-functionalized oxidized carbon black in brine.

### DETAILED DESCRIPTION

In the following description, certain details are set forth such as specific quantities, sizes, etc. so as to provide a thorough understanding of the present embodiments disclosed herein. However, it will be evident to those of ordinary skill in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

Referring to the drawings in general, it will be understood that the illustrations are for the purpose of describing particular embodiments of the disclosure and are not intended to be limiting thereto. Drawings are not necessarily to scale.

While most of the terms used herein will be recognizable to those of ordinary skill in the art, it should be understood, however, that when not explicitly defined, terms should be interpreted as adopting a meaning presently accepted by those of ordinary skill in the art. In cases where the construction of a term would render it meaningless or essentially meaningless, the definition should be taken from Webster's Dictionary, 3rd Edition, 2009. Definitions and/or interpretations should not be incorporated from other patent applications, patents, or publications, related or not, unless specifically stated in this specification or if the incorporation is necessary for maintaining validity.

The following definitions are set forth to aid in understanding of the various embodiments of the present disclosure. Terms in addition to those below are defined, as required, throughout the Detailed Description.

“Non-covalent association, non-covalently associated or not covalently bonded,” as used herein, refers to, for example, a molecular interaction between two or more moieties that does not include a covalent bond formed between the moieties. Non-covalent associations may include, for example, ionic interactions, acid-base interactions, hydrogen bonding interactions, π-stacking interactions, van der Waals interactions, adsorption, physisorption, self-assembly and sequestration.

“Insoluble in water or water-insoluble,” as used herein, refers to, for example, a condition in which a compound is substantially undissolved in a given quantity of water. As used herein, a substance will be considered to be water-insoluble if a stable solution having a concentration greater than about 0.1% (w/v) cannot be prepared in water.

“Analyte,” as used herein, refers to, for example, a moiety or condition being detected in an analysis. In an embodiment, analytes may include various chemical moieties being detected and/or quantified including, for example, organic compounds, inorganic compounds, ions, and heavy metals. In another embodiment, analytes may be a physical condition being measured including, for example, pressure, temperature, pH, redox potential and conductivity.

“Graphene,” as defined herein, refers to, for example, a single graphite sheet that is less than about 10 carbon layers thick.

“Graphene nanoribbons,” as defined herein, refer to, for example, single- or multiple layers of graphene that have an aspect ratio of greater than about 5, based on their length and their width.

“Amphiphilic,” as defined herein, refers to, for example, a material having both hydrophilic and hydrophobic domains. For example, oxidized carbon nanomaterials described herein are amphiphilic because they possess significant hydrophobic character, while at the same time they have polar functional groups that also confer significant hydrophilic character. Relative to non-oxidized carbon nanomaterials, the present oxidized carbon nanomaterials are significantly hydrophilic. Compositions presently disclosed herein containing amphiphilic nanomaterials may be made either hydrophobic or hydrophilic depending on the identity of the solubilizing groups appended to the amphiphilic nanomaterials.

The transport of hydrophobic organic molecules through porous media such as, for example, soil has been studied for many years to understand the percolation of pollutants into the environment. In isolation, hydrophobic organic molecules adsorb very strongly to nearly all types of soil. However, it has been observed that hydrophobic organic molecules disperse much more broadly in the environment than would be expected given their strong affinity for binding to soil. One possible explanation for this behavior is that organic macromolecules having amphiphilic characteristics may sequester small hydrophobic organic molecules and facilitate their transport by carrying them within in the organic macromolecule. This effect has been demonstrated in the laboratory with amphiphilic molecules such as, for example, cyclodextrin, showing highly efficient transport of the hydrophobic molecules. However, it has not been heretofore shown that small hydrophobic organic molecules can be selectively released from organic macromolecules, particularly to provide information regarding the environment to which the macromolecule/hydrophobic organic molecule conjugate has been exposed. Embodiments described herein demonstrate compositions and methods for selective transport and release of both non-covalently adsorbed and covalently bonded reporter molecules from water- and brine-soluble nanomaterials. By analyzing the compositions after release or uptake of reporter molecules, various inferences can be made regarding the environment to which the compositions have been exposed.

In various embodiments, compositions described herein include a transporter component and a signaling component. The transporter component includes an amphiphilic nanomaterial and a plurality of solubilizing groups covalently bonded to the amphiphilic nanomaterial. The signaling component includes a plurality of reporter molecules associated with the transporter component. At least a portion of the plurality of reporter molecules is releasable from the transporter component upon exposure to at least one analyte of interest.

In general, the amphiphilic nanomaterials of the present compositions can have different degrees of hydrophilicity/hydrophobicity depending on the extent of oxidation in the amphiphilic nanomaterial. The degree of hydrophilicity/hydrophobicity may be used to tailor the sequestration of reporter molecules. For example, in some embodiments, a more hydrophobic core may better sequester the reporter molecules. However, in other embodiments a more hydrophilic core may better sequester the reporter molecules.

Overall, the present compositions may be either hydrophobic or hydrophilic depending on the nature of the solubilizing groups. In the embodiments described herein, the solubilizing groups and the compositions are generally hydrophilic and release their associated reporter molecules upon exposure to hydrophobic environments or other related conditions. However, with a more hydrophilic nanomaterial core, the compositions may be solubilized with non-polar (hydrophobic) solubilizing groups (e.g., long chain alkanes or fatty acids). In these alternative embodiments, the compositions may sequester polar molecules and then release them upon exposure to hydrophilic environments (e.g., water).

In various embodiments, amphiphilic nanomaterials of the present disclosure include, for example, functionalized carbon nanotubes (e.g., oxidized carbon nanotubes having at least a plurality of carboxylic acids on their surface, including open ends and sidewalls), graphene oxide, graphene oxide nanoribbons and oxidized carbon black particles. In an embodiment, functionalized (oxidized) carbon nanotubes may be prepared by reacting a dispersion of carbon nanotubes with a mixture of fuming sulfuric acid and nitric acid. Single-, double- and multi-walled carbon nanotubes may be used to form the oxidized carbon nanotubes. In an embodiment, graphene oxide may be prepared by exfoliation of graphite as described in commonly-assigned international patent applications PCT/US09/30498 and PCT/US10/34905, each of which is incorporated herein by reference in its entirety. In an embodiment, oxidized graphene nanoribbons may be prepared as described in commonly-assigned United States Patent Application publication 2010/0105834, which is also incorporated herein by reference in its entirety. In an embodiment, oxidized carbon black may be prepared by a reaction of carbon black particles with an oxidizing agent such as, for example, KMnO₄ in a mixture of sulfuric acid and phosphoric acid, using a modification of the methods for oxidized graphene nanoribbons described in international patent application PCT/US10/34905. All of these amphiphilic nanomaterials are highly oxidized and contain various oxidized functionalities such as, for example, carboxylic acids, ketones, hydroxyl groups, and epoxides. These amphiphilic carbon nanomaterials are hydrophilic relative to most carbon nanomaterials, which are generally very hydrophobic.

In general, solubilizing groups of the present compositions are covalently bonded to the carboxylic acid groups of the aforementioned amphiphilic nanomaterials. However, covalent bonding of the solubilizing groups to the amphiphilic nanomaterials in any manner lies within the spirit and scope of the present disclosure. Reduction of the amphiphilic nanomaterials may be performed in which at least a portion of the oxidized functionalities remain after reduction. In particular, carboxylic acids are not easily reduced. Reduction of oxidized nanomaterials is more thoroughly described in international patent applications PCT/US09/30498 and PCT/US10/34905 and United States Patent Application publication 2010/0105834. Accordingly, in some embodiments, the oxidized amphiphilic nanomaterials may be reduced and then covalently bonded to the solubilizing groups. Alternatively, in other embodiments, the oxidized amphiphilic nanomaterials may be covalently bonded to the solubilizing groups and then reduced. In various embodiments, the combination of the amphiphilic nanomaterial and the plurality of solubilizing groups covalently bonded to the amphiphilic nanomaterial is water soluble. In other embodiments, the combination of the amphiphilic nanomaterial and the plurality of solubilizing groups covalently bonded to the amphiphilic nanomaterial is soluble in non-polar solvents including, for example, alkanes, aromatic solvents and oils. In some embodiments, the solubilizing groups are covalently bonded to the amphiphilic nanomaterials through oxidized edge functionality. In other embodiments, the solubilizing groups are covalently bonded to the amphiphilic nanomaterials away from their edge (e.g., on their basal plane).

Amphiphilic nanomaterials are not necessarily limited to the aforementioned examples, which are carbon-based. In some embodiments, the amphiphilic nanomaterials are inorganic nanoparticles such as, for example, silica nanoparticles. In some embodiments, suitable silica nanoparticles may contain a mixture of SiO₂ and an amine such as, for example, poly(allylamine). Such silica nanoparticles may be conveniently prepared with a highly uniform size distribution. In addition, silica nanoparticles containing poly(allylamine) include surface amino groups for binding that are not natively present in the carbon-based amphiphilic nanomaterials.

In other embodiments, amphiphilic nanomaterials of the present disclosure may be metal nanoparticles. In some embodiments, metal nanoparticles contain metals such as, for example, iron, silver, gold, tin, copper, nickel, palladium, platinum, magnesium, manganese, aluminum and alloys thereof. In general, metal nanoparticles of the present disclosure may contain any metallic element. In some embodiments, the metal nanoparticles may contain metal oxides such as, for example, aluminum oxide. In some embodiments, the metal nanoparticles are hollow. Hollow metal nanoparticles may collapse upon exposure to high pressure conditions. By engineering the size and shape of the hollow metal nanoparticles, the failure pressure needed to collapse the nanoparticles may be tuned to a predetermined value. The failure pressure may be used as a diagnostic tool to assay the maximum pressure encountered by the composition during use.

In various embodiments, solubilizing groups of the present disclosure are water-soluble polymers that are covalently bonded to the amphiphilic nanomaterials of the transporter component. Illustrative water-soluble polymers include, for example, poly(ethylene glycol) (PEG), poly(propylene glycol) (PPG), poly(vinyl alcohol) (PVA), poly(ethylene imine) (PEI), poly(acrylic acid), poly(hydroxyalkyl esters), polyvinyl pyrrolidone), PLURONICS (a registered trademark of BASF corporation for a block co-polymer of ethylene oxide and propylene oxide), saccharides, polysaccharides, carboxymethyl cellulose, and combinations thereof.

In some embodiments of the present compositions, the reporter molecules of the signaling component are covalently bonded to the transporter component. In other embodiments, the reporter molecules of the signaling component are not covalently bonded to the transporter component. When not covalently bonded to the transporter component, reporter molecules may be associated with the transporter component utilizing an interaction such as, for example, ionic interactions, acid-base interactions, hydrogen bonding interactions, n-stacking interactions, van der Waals interactions, adsorption, physisorption, self-assembly and sequestration. In some embodiments, the reporter molecules are hydrophobic (water-insoluble) molecules that are sequestered within the solubilizing groups of the transporter component. In some embodiments, the reporter molecules are hydrophilic (water-soluble) molecules that are sequestered within hydrophobic solubilizing groups covalently bonded to the transporter component. In various embodiments, the reporter molecules are not water soluble, but the transporter component is water soluble.

In some embodiments, the reporter molecules are all the same. In other embodiments, there are at least two different types of reporter molecules. In some embodiments, a first portion of the reporter molecules is covalently bonded to the transporter component and a second portion of the reporter molecules is not covalently bonded to the transporter component. The reporter molecules of the first portion and the reporter molecules of the second portion may be the same in some embodiments. However, in other embodiments, the reporter molecules of the first portion and the reporter molecules of the second portion may be different types of reporter molecules. In various embodiments, the first portion of reporter molecules and the second portion of reporter molecules may be operable to detect different analytes of interest when the first portion is covalently bonded to the transporter component and the second portion is not covalently bonded to the transporter component, even if the first portion and the second portion are formed from the same reporter molecules. Stated another way, the same reporter molecule may interact in a first manner with a first analyte of interest when covalently bonded to the transporter component and in a second manner with a second analyte of interest when not covalently bonded to the transporter component. In some embodiments, the first portion of reporter molecules and the second portion of reporter molecules are releasable from the transporter component at different rates. Such differential release rates may be diagnostic of the conditions to which the compositions have been exposed.

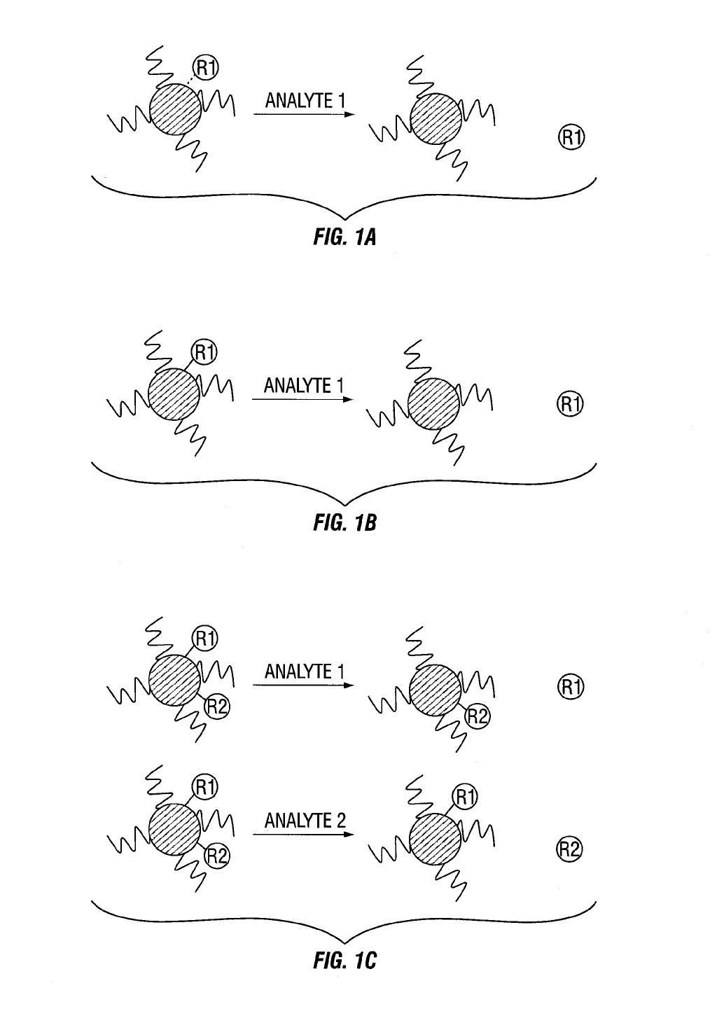

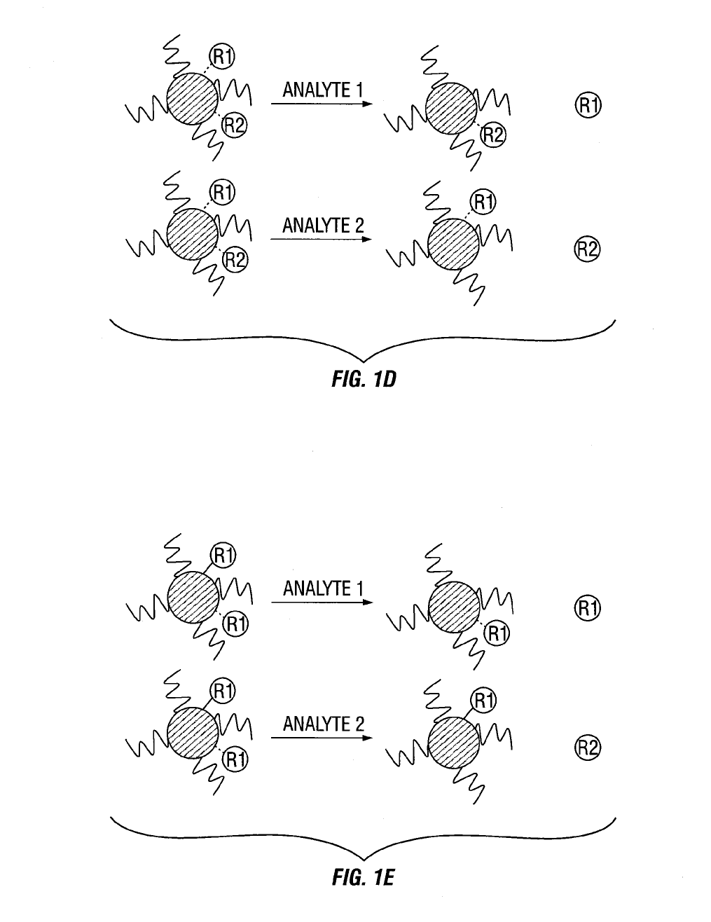

FIGS. 1A-1E present schematics showing some illustrative but non-limiting processes that the various compositions of the present disclosure may undergo upon exposure to one or more analytes or conditions of interest. In general, the processes of FIGS. 1A-1E demonstrate how a reporter molecule, particularly a hydrophobic reporter molecule, may be released from the present compositions in response to an encounter with one or more analytes or conditions of interest. In FIGS. 1A-1E, the central core represents any amphiphilic nanomaterial, and the wavy lines extending therefrom are a plurality of solubilizing groups. R1 and R2 represent a first type of reporter molecule and a second type of reporter molecule, respectively. Solid lines to R1 and R2 represent covalent bonds, and dashed lines represent non-covalent interactions with the amphiphilic nanomaterial and/or the solubilizing groups. In other various embodiments not depicted in FIGS. 1A-1E, reporter molecules may become associated or covalently bonded to the transporter component of the present compositions in response to an encounter with at least one analyte of interest. In still other embodiments not depicted in FIGS. 1A-1E, the amphiphilic nanomaterial and/or the reporter molecules may collapse in response to pressure. In any case, quantification of the reporter molecules provides information regarding the environment and conditions to which the compositions have been exposed.

As shown in FIG. 1A, a non-covalently bonded reporter molecule may be released from the present compositions in response to exposure to at least one analyte of interest. For example, in an embodiment, a hydrophobic reporter molecule sequestered in the solubilizing groups of the present compositions may be released when the composition is exposed to a hydrophobic analyte such as, for example, oil. Likewise, as shown in FIG. 1B, a covalent bond to a reporter molecule may be cleaved in the presence of at least one analyte, and the reporter molecule is thereafter released from the composition. Similarly, as shown in FIG. 1C, two different reporter molecules may be covalently bonded to the compositions and released preferentially upon exposure to two different analytes of interest. As shown in FIG. 1D, two different non-covalently bonded reporter molecules may be released at different rates, such that they can be used to detect two different analytes of interest. Finally, as shown in FIG. 1E, the same reporter molecule may be both non-covalently bonded and covalently bonded to the present compositions. The covalently-bonded reporter molecules are released from the compositions upon exposure to a first analyte, leaving the non-covalently bound reporter molecules sequestered. Likewise, the non-covalently bonded reporter molecules are released from the compositions upon exposure to a second analyte, leaving the covalently-bonded reporter molecules within the composition. As demonstrated by FIGS. 1C-1E, the present compositions may be used to detect various analytes or conditions simultaneously through careful choice of the reporter molecules. In general, any number of different types of reporter molecules can be included in the present compositions for detection of a number of different analytes or conditions.

In general, reporter molecules are any type of entity that is responsive in some way to the presence of at least one analyte of interest. Furthermore, the reporter molecules are generally capable of being assayed by various analytic methods either before or after being released from the present compositions as a means for quantifying the composition's interactions with the at least one analyte of interest. In some embodiments, the reporter molecules are released from the present compositions in the presence of at least one analyte of interest. In some embodiments, covalent bonds to the reporter molecules are broken in the presence of at least one analyte of interest. In other embodiments, covalent bonds are formed to the reporter molecules in the presence of at least one analyte of interest. In some embodiments, the reporter molecules are chemically transformed in the presence of at least one analyte of interest. For example, in some embodiments, the reporter molecules may be oxidized, reduced, rearranged or otherwise chemically reacted, thereby enabling transformed reporter molecules to be distinguished from non-transformed reporter molecules by some means (e.g. chemical or spectroscopic analyses).

In more specific embodiments, reporter molecules may be, for example, fluorescent dyes (e.g., 1,5-diphenyloxazole or fluorescein), UV-active molecules, radiolabeled molecules, isotopically enriched molecules (e.g., molecules having mass spectra distinct form non-isotopically enriched molecules), non-isotopically enriched molecules that are easily detectable by their mass spectra or other unique spectroscopic signature, metal nanoparticles and molecules that are sensitive to the presence of heavy metals (e.g., chelating ligands). In embodiments where the reporter molecule is a fluorescent dye, fluorescence is typically quenched when the fluorescent dye is in close proximity to the amphiphilic nanomaterial. However, upon release from the transporter component of the present compositions, the separation between the fluorescent dye and the amphiphilic nanomaterial becomes sufficient such that the fluorescence is no longer quenched. Hence, the detection of fluorescence from released fluorescent dye is indicative of the presence of at least one analyte of interest. Other reporter molecules may be detected and analyzed while either sequestered in the compositions or after being released from the compositions.

In some embodiments, the concentration of reporter molecules decreases in the present compositions in response to an encounter with at least one analyte of interest. In other embodiments, the concentration of reporter molecules increases in the present compositions in response to at least one analyte of interest. In embodiments where the concentration of reporter molecules increases, the reporter molecule is part of the at least one analyte of interest. Stated another way, when the concentration of reporter molecules increases, the at least one analyte of interest is taken up and sequestered by the present compositions. In some embodiments, change in a spectral property represents a means through which the reporter molecules indicate an encounter with at least one analyte of interest. For example, in an embodiment, reporter molecules that are covalently bonded to the present compositions may undergo a spectral shift upon having their covalent bonds broken in the presence of at least one analyte of interest. In other embodiments, the reporter molecules may be used in a barcode fashion to follow sequential change in the concentration of at least one analyte of interest over the course of time.

In some embodiments, the present compositions may be used in a barcoding fashion as described hereinafter. For example, a composition containing a transporter component and a signaling component may be provided according to the previously described embodiments. For barcoding applications, the composition further includes a cleavable or non-cleavable identification tag such as, for example, a radioisotope tag, a fluorescent dye or any other molecule having an easily identified spectroscopic signature. Thereafter, the composition containing the identification tag is injected into a geological formation, and the geological formation is assayed for the presence of the identification tag over the course of time. Over the course of time, additional compositions containing different identification tags may be subsequently injected into the geological formation. The unique signaling properties of the identification tag may be used to correlate the location and date of injection for each composition with the location and date of recovery of the composition from the production hole. By having the injection and recovery locations and dates, along with signaling information from the reporter molecules, a rough topology of the analyte environment within the geological formation can be mapped. For example, for a given composition containing identification tag injected at time=0, some of the composition may be recovered at a first time t₁ and some of the composition may be recovered at a second time t₂. If the composition was exposed to little analyte of interest (e.g., petroleum) during t₁ but significantly more analyte of interest during t₂, based on the assay of the reporter molecules contained therein, one could infer that there was a higher concentration of the analyte of interest at geological bands looping far from the injection and exit holes or that the composition recovered at t₂ was progressing through much smaller and slower moving pores between the entry and exits points.

In general, the at least one analyte of interest may be any substance or condition to be sensed in a porous substance or a liquid environment. In some embodiments, the at least one analyte of interest is part of a geological formation such as, for example, an oilfield. In some embodiments, the at least one analyte of interest is a hydrophobic substance such as, for example, petroleum. In other embodiments, the at least one analyte of interest is a sulfur-containing compound such as, for example, hydrogen sulfide or thiols. In some embodiments, the present compositions are responsive to at least one physical property of an aqueous environment such as, for example, presence or absence of an analyte of interest in the aqueous environment, relative abundance of water, relative abundance of hydrophobic compounds, electrolyte concentration, pressure, temperature, pH, redox potential and combinations thereof.

One desirable feature of the present compositions is their ability to flow through porous environments. In general, the present compositions are of nanoscale size in at least one dimension and have a size between about 40 nm and about 300 nm. However, in other embodiments, the compositions have a size less than about 1000 nm. At these sizes, the present compositions are operable to flow through very small pores in a porous medium. In various embodiments, the present compositions are operable to flow through a porous medium such as, for example, soil, rock formations, and oil-containing geological formations. In addition, another desirable feature of the present compositions is their stability in aqueous salt solutions. Aqueous salt solutions are commonly encountered in geological formations, particularly those used for oil production. Hence, the present compositions have the ability to flow through porous media in the presence of an aqueous salt solution such as, for example, brine. In other embodiments, the present compositions may flow through aqueous streams such as, for example, surface water streams and waste streams.

In some embodiments of the compositions, the transporter component includes an amphiphilic nanomaterial and a plurality of solubilizing groups covalently bonded to the amphiphilic nanomaterial, and the signaling component includes a first plurality of reporter molecules covalently bonded to the transporter component. At least a portion of the first plurality of reporter molecules is cleavable from the transporter component upon exposure to at least one analyte of interest.

In general, the reporter molecules are covalently bonded to the transporter component by a covalent bond that is readily cleavable in the presence of at least one analyte of interest. However, in alternative embodiments, the covalent bond to the reporter molecules may be more robust and not readily cleavable (e.g., amides and carbamates). In some embodiments, the covalent bond between the reporter molecules and the transporter component is an ester bond. In other embodiments, the covalent bond between the reporter molecules and the transporter component is a disulfide bond. Each of these types of covalent bonds is cleavable upon exposure to conditions or analytes of interest commonly found in geological formations. For example, ester bonds are labile in the presence of acid and especially base, and disulfide bonds are labile in the presence of sulfur-containing compounds such as hydrogen sulfide and thiols. Hence, when reporter molecules are covalently bonded to the present compositions by ester or disulfide covalent bonds, the compositions may be used to sense conditions of pH and the presence of sulfur-containing compounds, respectively.

In some embodiments, the compositions further include a second plurality of reporter molecules that are covalently bonded to the transporter component. At least a portion of the second plurality of reporter molecules is cleavable from the transporter component upon exposure to at least one analyte of interest. Furthermore, the first plurality of reporter molecules and the second plurality of reporter molecules are operable to detect different analytes of interest. An embodiment of such compositions is presented schematically in FIG. 1C.

In some embodiments, the compositions further include a second plurality of reporter molecules that are not covalently bonded to the transporter component. At least a portion of the second plurality of reporter molecules is releasable from the transporter component upon exposure to at least one analyte of interest. Furthermore, the first plurality of reporter molecules and the second plurality of reporter molecules are operable to detect different analytes of interest. An embodiment of such compositions is presented schematically in FIG. 1E. In some embodiments, the first plurality of reporter molecules and the second plurality of reporter molecules are the same. In other embodiments, the first plurality of reporter molecules and the second plurality of reporter molecules are different.

In other various embodiments, compositions of the present disclosure include a transporter component having an amphiphilic nanomaterial and a plurality of solubilizing groups covalently bonded to the amphiphilic nanomaterial. The transporter component is operable to become covalently bonded to a reporter molecule upon exposure to at least one analyte of interest. In some embodiments, the reporter molecule is the at least one analyte of interest. Stated another way, in some embodiments, the present compositions may become covalently bonded to at least one analyte of interest, where the at least one analyte of interest serves as a reporter molecule once covalently bonded to the transporter component. In other embodiments, the compositions further include a plurality of reporter molecules that are non-covalently associated with the transporter component, but upon exposure to at least one analyte of interest, at least a portion of the plurality of reporter molecules is operable to become covalently bonded to the transporter component. Stated another way, in some embodiments, exposure of the present compositions to at least one analyte of interest may facilitate covalent bond formation to a previously non-covalently sequestered reporter molecule in the transporter component.

In still other various embodiments, compositions of the present disclosure include a transporter component having an amphiphilic nanomaterial that is collapsible at a predetermined pressure and a plurality of solubilizing groups that are covalently bonded to the amphiphilic nanomaterial. In various embodiments, such collapsible amphiphilic nanomaterials include metal nanoparticles such as, for example, hollow nanoparticles or nanoshells. In some embodiments, such compositions further include a signaling component containing a plurality of reporter molecules that is associated with the transporter component, wherein at least a portion of the reporter molecules is releasable from the transporter component upon exposure to at least one analyte of interest. Therefore, some embodiments of the compositions are capable of sensing pressure and at least one other analyte of interest. In some embodiments, the plurality of reporter molecules is covalently bonded to the transporter component. In other embodiments, the plurality of reporter molecules is not covalently bonded to the transporter component.

In other various embodiments, methods for using the above compositions are described herein. In some embodiments, the methods include providing a composition having a transporter component and a signaling component, exposing the composition to a liquid medium containing at least one analyte of interest and recovering the composition from the liquid medium after exposing. The transporter component includes an amphiphilic nanomaterial and a plurality of solubilizing groups covalently bonded to the amphiphilic nanomaterial. The signaling component includes a plurality of reporter molecules non-covalently associated with the transporter component. At least a portion of the plurality of reporter molecules is released from the transporter component upon exposure to the at least one analyte of interest. The plurality of reporter molecules are present in a first concentration in the composition prior to exposing and in a second concentration after exposing. The methods further include assaying the composition to determine the second concentration.

In some embodiments of the methods, the ratio of the second concentration to the first concentration can be correlated with an amount of the at least one analyte of interest in the liquid medium. For example, in an embodiment, a standard calibration curve of the present compositions containing variable concentrations of the various reporter molecules may allow such a correlation to be made. In some embodiments, the methods further include assaying the liquid medium for the portion of the plurality of reporter molecules released from the transporter component. For example, in embodiments in which the reporter molecules are fluorescent dyes, the fluorescence may be more readily observed from free reporter molecules in the liquid medium, rather than fluorescent dyes in the compositions due to possible fluorescence quenching by the amphiphilic nanomaterial. Hence, the reporter molecules may convey information about the environment to which the compositions have been exposed, either when the reporter molecules are part of the compositions or as free reporter molecules in the liquid medium.

In various embodiments, liquid media suitable for practicing the present methods include, for example, a geological formation containing a liquid medium, a wastewater source, a ground water source, and a surface water source. In various embodiments, the liquid medium is flowing. In some embodiments, the liquid medium is adsorbed on to a solid surface such as, for example, a rock surface.

In general, in methods for assaying a geological formation such as, for example, an oil well, the compositions are released downhole via injection, which is followed thereafter by injection of water or brine. The compositions move through the geological formation until the water or brine injection terminates. After a variable time, the flow is reversed such that the compositions are then pulled back through the injection well or a production well for analysis. Samples are collected and analyzed by standard characterization techniques. The residence time of the compositions in the geological formation is dependent on a number of factors including, for example, the time before the flow is reversed and the distance the compositions initially travel during injection. During their time in the geological formation, the compositions lose hydrophobic reporter molecules to any hydrophobic media contained therein such as, for example, petroleum. Given the residence time, as well as the known well temperature, the amount of hydrophobic reporter molecules lost can be diagnostic of the amount of petroleum contained in the geological formation.

In still other various embodiments, methods of the present disclosure include providing a composition having a transporter component and a signaling component, exposing the composition to a liquid medium containing at least one analyte of interest and recovering the composition from the liquid medium after exposing. The transporter component includes an amphiphilic nanomaterial and a plurality of solubilizing groups covalently bonded to the amphiphilic nanomaterial. The signaling component includes a plurality of reporter molecules covalently bonded to the transporter component. At least a portion of the plurality of reporter molecules is cleaved from the transporter component upon exposure to the at least one analyte of interest. The plurality of reporter molecules is present in a first concentration in the composition prior to exposing and in a second concentration after exposing. The methods further include assaying the composition to determine the second concentration.

In some embodiments of the methods, the reporter molecules are covalently bonded to the transporter component by an ester bond. In other embodiments of the methods, the reporter molecules are covalently bonded to the transporter component by a disulfide bond. In embodiments in which the reporter molecules are covalently bonded by a disulfide bond, the at least one analyte of interest may be a sulfur-containing compound such as, for example, a thiol or hydrogen sulfide. Ester and disulfide bonds are readily cleavable and may be used when release of the reporter molecules from the compositions is desired. However, in some embodiments, it may be desirable to have the reporter molecules more robustly bound to the transporter component. For example, if the reporter molecules are to be oxidized, reduced, rearranged, reacted or otherwise chemically transformed, more robust covalent bonds may be desirable. In such embodiments, covalent bonds such as, for example, amides and carbamates may be used.

In some embodiments, the reporter molecules are released from the transporter component upon being cleaved. In some embodiments of the methods, the ratio of the second concentration to the first concentration can be correlated with an amount of the at least one analyte of interest in the liquid medium. In some embodiments, the method further include assaying the liquid medium for the portion of the plurality of reporter molecules cleaved from the transporter component.

In some embodiments, liquid media suitable for practicing the present methods include, for example, a geological formation containing a liquid medium, a wastewater source, a ground water source, and a surface water source. In various embodiments, the liquid medium is flowing.

In other various embodiments, methods of the present disclosure include providing a composition having a transporter component containing an amphiphilic nanomaterial and a plurality of solubilizing groups covalently bonded to the amphiphilic nanomaterial, exposing the composition to a liquid medium containing at least one analyte of interest, recovering the composition from the liquid medium after exposing, and assaying the composition to determine the concentration of the at least one analyte of interest in the composition. At least a portion of the at least one analyte of interest becomes associated with the transporter component during exposing.

In some embodiments of the methods, the concentration of the at least one analyte of interest in the composition can be correlated with an amount of the at least one analyte of interest in the liquid medium. Hence, in some embodiments of the methods, the compositions may extract at least one analyte of interest from the composition to provide information about conditions within the liquid medium. In some embodiments, suitable liquid media for practicing the present methods may include, for example, a geological formation containing a liquid medium, a wastewater source, a ground water source, and a surface water source.

In still additional embodiments, methods of the present disclosure include providing a composition having a transporter component and a signaling component, injecting the composition into a geological formation containing at least one analyte of interest, and recovering the composition from the geological formation after a period of time. The transporter component includes an amphiphilic nanomaterial and a plurality of solubilizing groups covalently bonded to the amphiphilic nanomaterial. The signaling component includes a plurality of reporter molecules associated with the transporter component. At least a portion of the plurality of reporter molecules is released from the transporter component upon exposure to the at least one analyte of interest. The plurality of reporter molecules are present in a first concentration in the composition prior to injection and in a second concentration after being recovered. The methods further include assaying the composition to determine the second concentration. The ratio of the second concentration to the first concentration can be correlated with an amount of the at least one analyte of interest in the geological formation.

In further embodiments, the methods further include assaying the geological formation for the portion of the plurality of reporter molecules released from the transporter component. In some embodiments of the methods, the composition is injected into the geological formation in a first location and recovered in a second location (e.g., injection wells and production wells). However, in other embodiments, the composition is injected into the geological formation and recovered from the geological formation in the same location. In some embodiments, the composition is injected while dissolved in a substantially aqueous medium (>50% water). In other embodiments, the composition is injected while dissolved in a substantially organic medium (>50% organic solvent).

In still other various embodiments, methods of the present disclosure include providing a first composition containing a transporter component containing an amphiphilic nanomaterial and a plurality of solubilizing groups covalently bonded to the amphiphilic nanomaterial and a signaling component containing a first identification tag covalently bonded to the transporter component, injecting the first composition into a geological formation, providing a second composition containing a transporter component containing an amphiphilic nanomaterial and a plurality of solubilizing groups covalently bonded to the amphiphilic nanomaterial and a signaling component containing a second identification tag covalently bonded to the transporter component, injecting the second composition into the geological formation after a first period of time separating the injection of the first composition and the injection of the second composition, and assaying the geological formation for the presence of the first composition and the second composition.

In various embodiments, a concentration of the first composition and a concentration of the second composition in the geological formation are diagnostic of physical changes that occur in the geological formation over the first period of time. For example, a change in concentration of the identification tag in the recovered second composition compared to the first recovered composition would be indicative of a significant change in physical conditions that occurred within the geological formation. Similarly, in some embodiments, a time taken for the first composition to be detected and a time taken for the second composition to be detected can be correlated with a distance that the first composition and the second composition travelled in the geological formation. Again, a change in distance travelled by the first composition compared to that travelled by the second composition may be indicative of a change in physical conditions within the geological formation. Likewise, the concentrations and transit times may be diagnostic of an internal structure of the geological formation.

In general, the methods can be extended to include any number of compositions containing an identification tag and any number of time periods over which assays are to be made. In some embodiments, the methods further include providing a third composition containing a transporter component containing an amphiphilic nanomaterial and a plurality of solubilizing groups covalently bonded to the amphiphilic nanomaterial and a signaling component containing a third identification tag covalently bonded to the transporter component, injecting the third composition into the geological formation after a second period of time separating the injection of the second composition and the injection of the third composition, and assaying the geological formation for the presence of the first composition, the second composition and the third composition. Concentrations of the first composition, the second composition and the third composition are diagnostic of physical changes that occur in the geological formation over the second period of time. In these and other embodiments, the present compositions and methods may be used in a barcode fashion, as the relative abundances of the compositions recovered after various periods of time are unique to conditions encountered in the geological formation during a given period of time.

In some embodiments, any one or more of the first composition, the second composition and the third composition may contain a signaling component associated with the transporter component. In such embodiments, the reporter molecules may be used to assay at least one analyte of interest as described hereinabove, while the identification tags may be used to define when and where the compositions were injected into the geological formation.

In various embodiments, the identification tags covalently bonded to the compositions may include, without limitation, fluorescent dyes, radiolabelled molecules and isotopically labeled molecules. Each of these identification tags offer very low detection limits and unique spectroscopic signatures compared to naturally occurring molecules in a geological formation. In some embodiments, fluorescent dyes may be covalently bonded to the transporter component by a covalent bond that is not easily cleavable under conditions encountered in the geological formation. After the compositions are recovered, however, the covalent bonds to the fluorescent dyes may be cleaved, if desired, particularly to overcome fluorescence quenching. Otherwise, the fluorescent dyes may be left covalently bonded to the compositions while being assayed. In other embodiments, it may be more advantageous to have a fluorescent dye that is cleavable in the geological formation upon exposure to at least one analyte of interest.

In still further embodiments, the methods include providing a composition containing a transporter component containing an amphiphilic nanomaterial and a plurality of solubilizing groups covalently bonded to the amphiphilic nanomaterial and a signaling component containing a plurality of reporter molecules associated with the transporter component and an identification tag covalently bonded to the transporter component, injecting the composition into a geological formation in a first location, recovering the composition from the geological formation in a second location over a period of time, and analyzing the composition recovered from the second location.

In some embodiments, the time between injecting and recovering the composition can be correlated with an internal structure of the geological formation. In other embodiments, a concentration of the plurality of reporter molecules can be correlated with a concentration of at least one analyte of interest within the geological formation.

Compositions and methods described herein may be used in a wide variety of applications. In some embodiments, the present compositions and methods may be used in oil production to determine if a putative or existing oil well contains or still contains significant amounts of hydrocarbons (a hydrophobic material). For example, injection of the present compositions into an oil well containing large amounts of hydrocarbons results in recovery of compositions having relatively low quantities of sequestered reporter molecules. In contrast, injection of the present compositions into an oil well containing mostly non-hydrocarbon material will result in recovery of a relatively intact composition, indicating that the well is not an ideal site for production. Similarly, the compositions may be used to determine, for example, if any petroleum in a well is of high sulfur or heavy metal content by using reporter molecules that are sensitive for each of these materials. As described hereinabove, detection of sulfur and heavy metals may be conducted simultaneously or separately from the assay for identification of petroleum in the well.

In addition, in some embodiments, the reporter molecules may have functional capabilities upon being released in the presence of at least one analyte of interest. For example, in some embodiments, reporter molecules released after interaction with petroleum may be functional to alter the interaction of the petroleum with its subsurface environment and increase the rate or total amount of production. In other embodiments, reporter molecules released upon exposure to a soil contaminant may actively remediate or decontaminate the soil.

When the present compositions are initially lacking a reporter molecule, they may uptake a reporter molecule upon exposure to at least one analyte of interest. In this regard, the present compositions may also be used in environmental remediation to treat a porous substance (e.g., soil) or a liquid medium containing one or more contaminants that may become sequestered by the transporter component of the present compositions. In an embodiment, the one or more contaminants being sequestered by the present compositions may also become covalently bonded to the compositions. In another embodiment, the present compositions may be used to detect and concentrate a trace contaminant in a soil sample or a water sample, for example. In the process of detecting and concentrating the trace contaminant, the soil or water sample is purified or remediated.

In summary, the present compositions and methods may be used for analysis and/or remediation of any flow-based process. Such flow-based processes can occur in porous media such as, for example, soils and geological formations, including oil fields and ground water streams. Flow-based processes can also include both manmade streams and natural streams such as, for example, industrial streams, waste water treatment streams, and surface water streams.

### EXPERIMENTAL EXAMPLES

The following examples are provided to more fully illustrate some of the embodiments disclosed hereinabove. It should be appreciated by those of ordinary skill in the art that the methods disclosed in the examples that follow represent techniques that constitute illustrative modes for practice of the disclosure. Those of ordinary skill in the art should, in light of the present disclosure, appreciate that many changes can be made in the specific embodiments that are disclosed and still obtain a like or similar result without departing from the spirit and scope of the disclosure.

### Example 1

### Syntheses of Amphiphilic Nanomaterials

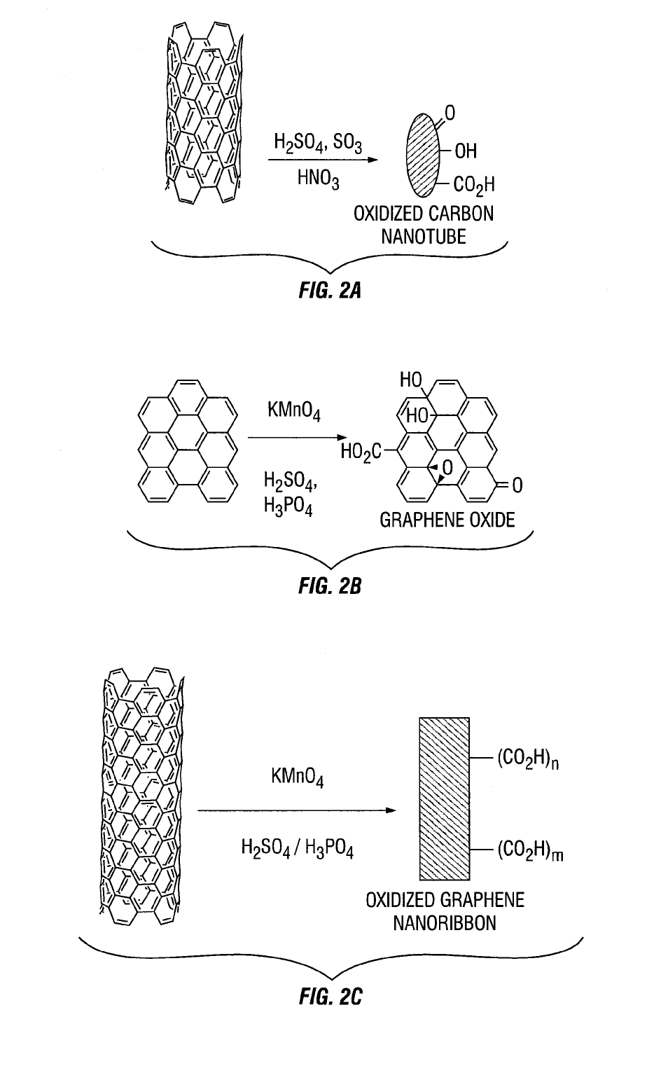

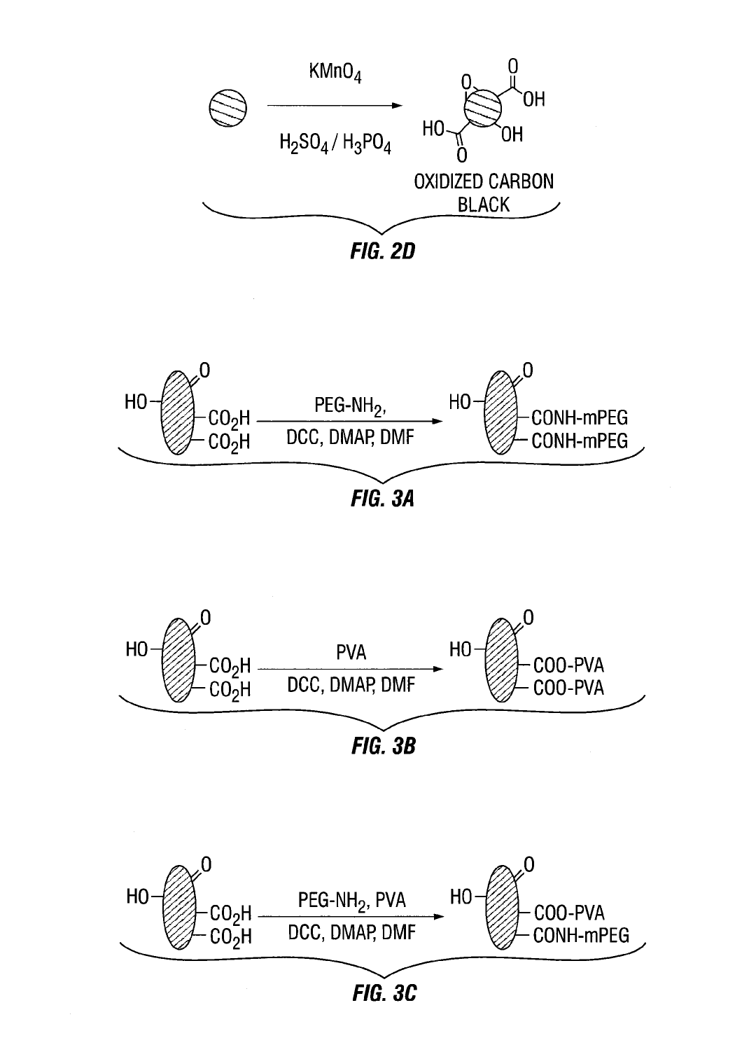

Amphiphilic nanomaterials of the present experimental examples include oxidized carbon nanotubes, graphene oxide, oxidized graphene nanoribbons, and oxidized carbon black. Oxidized carbon nanotubes were synthesized by oxidizing single-walled carbon nanotubes with a mixture of fuming sulfuric acid and nitric acid. Graphene oxide was synthesized by oxidative exfoliation of graphite. Graphene nanoribbons were synthesized by the oxidation of multi-walled carbon nanotubes using KMnO₄ in the presence of sulfuric acid and phosphoric acid. Oxidized carbon black was likewise synthesized through oxidation of carbon black using KMnO₄ in the presence of sulfuric acid and phosphoric acid. FIGS. 2A-2D show illustrative reactions for synthesizing the amphiphilic nanomaterials. The structures of the amphiphilic nanomaterials are illustrative of the functionality that might be introduced upon oxidation, and the particular placement of functional groups should not be taken as limiting. In general, the oxidation introduces a plurality of carboxylic acid groups that can be further functionalized with solubilizing groups, as described hereinafter. Further details regarding the synthesis of oxidized carbon nanotubes, graphene oxide and oxidized graphene nanoribbons may be found in international patent applications PCT/US08/78776, PCT/US09/30498 and PCT/US10/34905 and United States Patent Application Publication 2010/0105834, all of which were previously incorporated by reference herein.

### Example 2

### General Procedure for Covalently Bonding Solubilizing Groups to Amphiphilic Nanomaterials

Solubilizing groups of the present examples include poly(ethylene glycol) (PEG) and poly(vinyl alcohol) (PVA). These water-soluble polymers were attached to the amphiphilic nanomaterials through formation of amide or ester bonds via free carboxylic acid groups on the surface of the amphiphilic nanomaterials, generally in the presence of dicyclohexylcarbodiimide (DCC) and dimethylaminopyridine (DMAP) in dimethylformamide (DMF) solvent. Formation of amide bonds to oxidized carbon nanotubes and oxidized graphene nanoribbons is generally described in PCT/US08/78776 and United States Patent Application Publication 2010/0105834, each of which was previously incorporated herein by reference. Formation of ester bonds was conducted under similar conditions. FIGS. 3A-3C show illustrative reactions for covalently bonding solubilizing groups to oxidized carbon nanotubes and other oxidized nanomaterials. Similar reactions can be performed with graphene oxide, oxidized graphene nanoribbons and oxidized carbon black to covalently bond solubilizing groups to these amphiphilic nanomaterials. As shown in FIG. 3C, a mixture of solubilizing groups may be covalently bonded to the amphiphilic nanomaterials.

### Example 3

### Sequestration of 2,5-Diphenyloxazole (PPO) in an Oxidized Carbon Nanotube Transporter Component

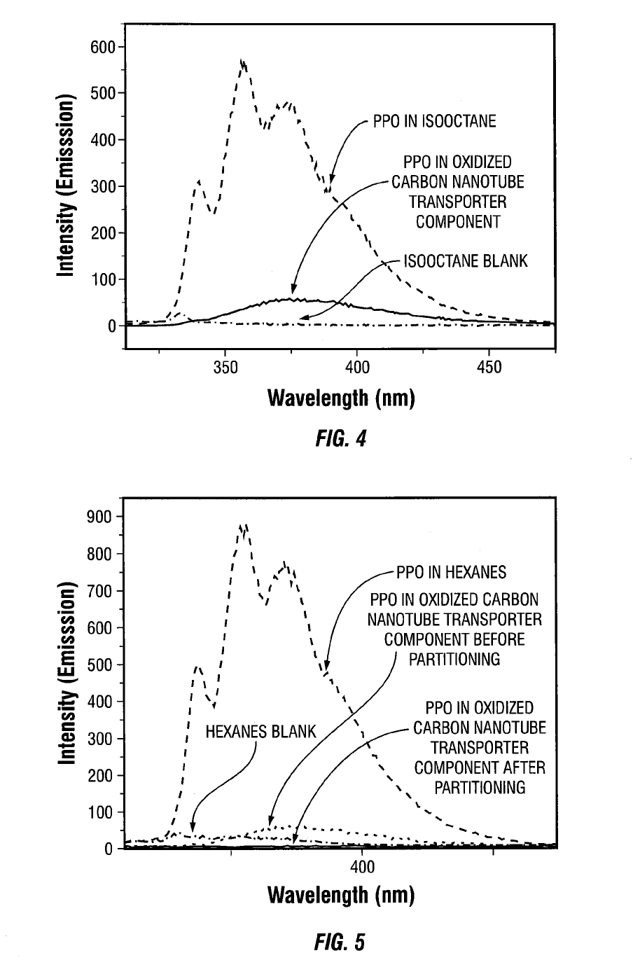

A transporter component was synthesized from oxidized carbon nanotubes and functionalized with poly(ethylene glycol) as described in Examples 1 and 2. To a 10 mL solution of this transporter component (10 mg of carbon per liter, 8 μmol) was added a solution of 10 mg of 2,5-diphenyloxazole (PPO) (45 μmol) in 10 mL tetrahydrofuran (THF). The solution was stirred for 16 hours and then concentrated to ˜8 mL to remove the THF. The resulting suspension was then eluted through a PD-10 desalting column containing Sephadex G-25 stationary phase (GE Healthcare) to remove any weakly bound PPO. FIG. 4 shows illustrative fluorescence spectra (280 nm excitation) of PPO in isooctane, PPO sequestered in an oxidized carbon nanotube transporter component and an isooctane blank. As shown in FIG. 4, the fluorescence intensity was reduced and shifted to slightly longer wavelengths when the PPO was sequestered in the oxidized carbon nanotube transporter component. The decreased fluorescence intensity is likely due to quenching induced by the oxidized carbon nanotube, which is known to occur when a fluorophore is in close proximity to a carbon nanotube. The oxidized carbon nanotube transporter component alone showed essentially no fluorescence (not shown).

### Example 4

### Release of PPO from an Oxidized Carbon Nanotube Transporter Component in the Presence of an Alkane

The solution of Example 3 obtained from the desalting column (5 mL) was stirred with hexanes (5 mL) for 15 minutes. Thereafter, the hexane phase and the aqueous phase were partitioned, and their fluorescence spectra were analyzed. FIG. 5 shows illustrative fluorescence spectra (280 nm excitation) of PPO extracted into the hexanes phase, PPO sequestered in the oxidized carbon nanotube transporter component before and after partitioning, and a hexanes blank. As shown in FIG. 5, essentially all of the PPO was extracted from the oxidized carbon nanotube transporter component into the hexanes phase. These results demonstrate that the present compositions can stabilize hydrophobic molecules in an aqueous environment and thereafter release the hydrophobic molecules in the presence of a lipophilic (hydrophobic) environment.

### Example 5

### Sequestration of Phenanthrene in Oxidized Carbon Nanotube and Oxidized Graphene Nanoribbon Transporter Components

Transporter components functionalized with poly(ethylene glycol) were synthesized from oxidized carbon nanotubes and oxidized graphene nanoribbons as described in Examples 1 and 2. For each transporter component, sequestration of phenanthrene was conducted as follows: A solution of the transporter component (5 mL, 50 mg of carbon per liter, 0.02 mmol) was stirred with phenathrene (2 mg, 0.01 mmol) in THF (5 mL) for 16 hours. The solution was concentrated to ˜3 mL to remove THF. The resulting suspension was then eluted through a PD-10 desalting column containing Sephadex G-25 stationary phase (GE Healthcare) to remove any weakly bound phenanthrene. Each solution was then diluted to 5 mL with H₂O.

### Example 6

### Release of Phenanthrene from an Oxidized Carbon Nanotube Transporter Component and an Oxidized Graphene Nanoribbon Transporter Component in the Presence of an Alkane

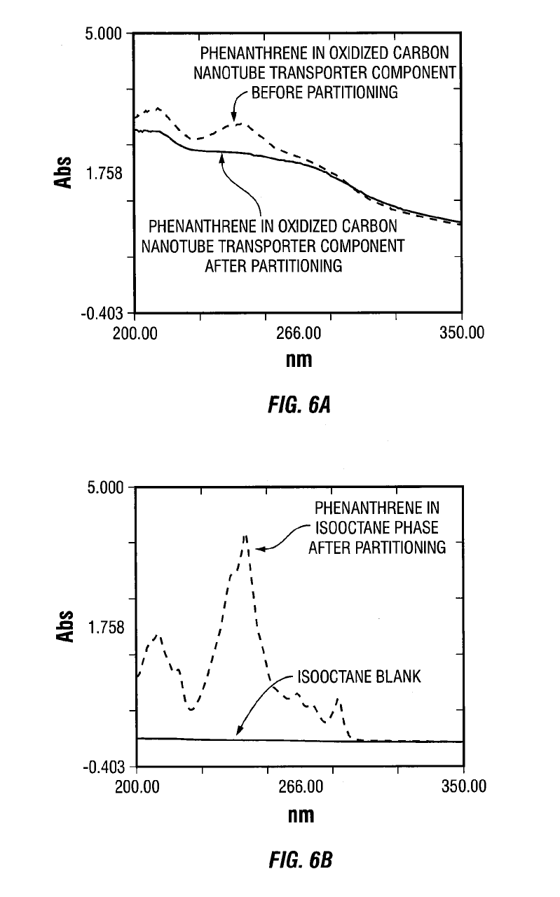

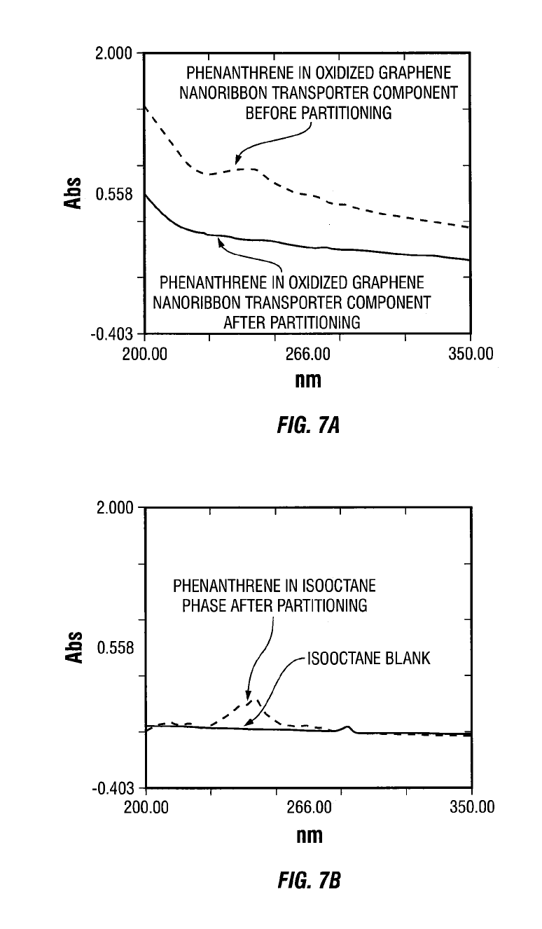

For each transporter component, 3 mL of the solution prepared in Example 5 was stirred with 3 mL of isooctane for 15 minutes. The phases were then separated and analyzed by UV-VIS spectroscopy. FIGS. 6A and 6B show illustrative UV-VIS spectra of phenanthrene in the isooctane extract and in the oxidized carbon nanotube transporter component before and after partitioning. FIGS. 7A and 7B show illustrative UV-VIS spectra of phenanthrene in the isooctane extract and in the oxidized graphene nanoribbon transporter component before and after partitioning. As shown in FIGS. 6A and 7A, a characteristic absorption for phenanthrene near 251 nm was present in each transporter component before partitioning. After partitioning, the characteristic absorption peak for phenanthrene was no longer present. As shown in FIGS. 6B and 7B, the characteristic absorption peak for phenathrene appeared in the isooctane phase after partitioning. These results again indicate that the present compositions can stabilize hydrophobic molecules in an aqueous environment and thereafter release the hydrophobic molecules in the presence of a lipophilic (hydrophobic) environment.

### Example 7

### Detection of Isooctane in Isooctane-Loaded Sand by an Oxidized Carbon Nanotube Transporter Component

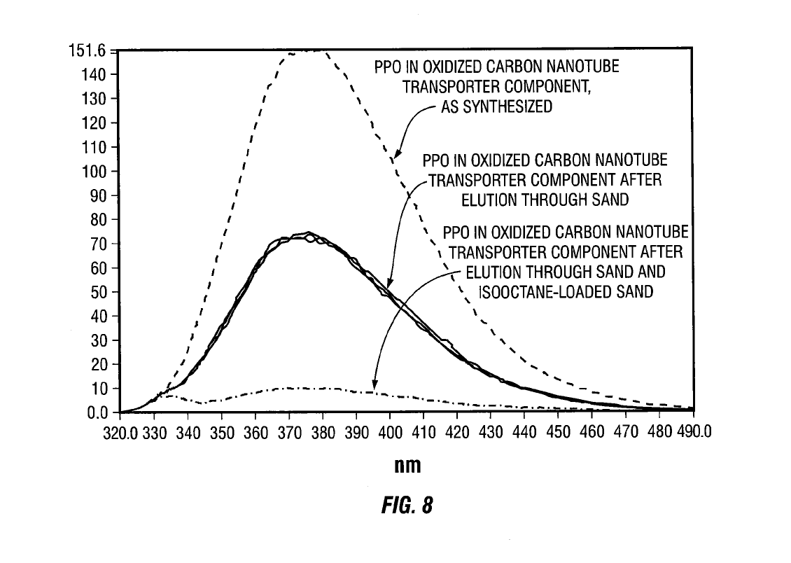

An oxidized carbon nanotube transporter component functionalized with poly(ethylene glycol) was synthesized as described in Examples 1 and 2. Sequestration of PPO in the oxidized carbon nanotube transporter component was conducted as described in Example 3. The composition containing sequestered PPO was eluted through a column of clean sand 3 times. After three passes through clean sand, the composition containing sequestered PPO was then eluted through a column containing sand treated with isooctane. FIG. 8 shows illustrative fluorescence spectra of PPO sequestered in a carbon nanotube transporter component as prepared, after passage through a column of clean sand and after passage through a column of isooctane-treated sand. As shown in FIG. 8, strong PPO fluorescence was observed in the composition. Although there was some decrease in the PPO fluorescence after the first passage through the column of clean sand, there was no further decrease thereafter. The decreased fluorescence intensity was likely due to weakly bound PPO that was removed upon elution. After passage through the column of isooctane-loaded sand, the fluorescence intensity dropped dramatically. This result demonstrates that the PPO was extracted from the oxidized carbon nanotube transporter component in the presence of the hydrophobic compound isooctane. These results indicate that the present compositions may be used to detect oil or other hydrophobic compounds when the hydrophobic compounds are contained in a porous medium.

### Example 8

### Detection of Oil in Sandstone and Dolomite Using an Oxidized Carbon Nanotube Transporter Component Containing a ¹⁴C Radiotracer

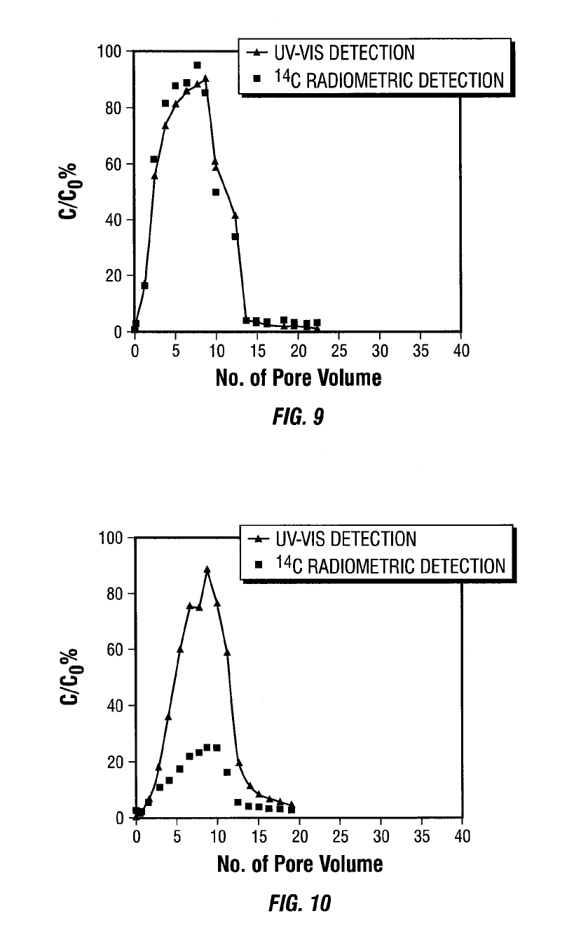

An oxidized carbon nanotube transporter component functionalized with a mixture of poly(ethylene glycol) and poly(vinyl alcohol) was synthesized as described in Examples 1 and 2. Sequestration of a ¹⁴C-radiolabelled aromatic compound was conducted analogously to the process described in Example 3. Radiotracer methods were used in the analysis of this example due to the ease of quantitation. After synthesis, the compositions were flowed through sandstone and dolomite core samples in a manner similar to that described in Example 7. FIG. 9 shows illustrative elution profiles from sandstone of the oxidized carbon nanotube transporter component containing a sequestered ¹⁴C aromatic compound as analyzed radiometrically and the oxidized carbon nanotube transporter component alone as analyzed by UV-VIS spectroscopy. As shown in FIG. 9, concentrations of the eluents were essentially identical, indicating that the sandstone did not contain residual oil. FIG. 10 shows illustrative elution profiles from dolomite of the oxidized carbon nanotube transporter component containing a sequestered ¹⁴C aromatic compound as analyzed radiometrically and the oxidized carbon nanotube transporter component alone as analyzed by UV-VIS spectroscopy. As shown in FIG. 10, the concentration of the eluent of the radiolabelled molecule was much lower than that of the oxidized carbon nanotube transporter component, indicating that some of the ¹⁴C aromatic compound was extracted by residual oil in the sample. The apparent differences between the two analyses is most likely due to the fairly small amount of oil still present in the dolomite sample and the high sensitivity of the radiometric method. In FIGS. 9 and 10, the vertical axis is the percentage of the starting activity or absorbance, and the horizontal axis is the number of pore volumes eluted.

### Example 9

### Synthesis of an Oxidized Carbon Nanotube Transporter Component Containing a Disulfide-Bonded Fluorescent Dye

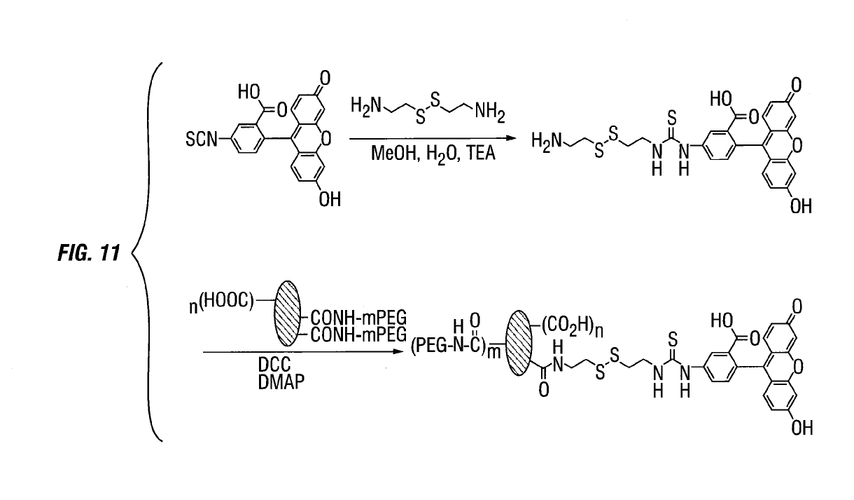

FIG. 11 shows a schematic of a representative synthetic route used to prepare an illustrative oxidized carbon nanotube transporter component containing a disulfide-bonded fluorescent dye. The synthesis of an oxidized carbon nanotube transporter component covalently bonded via a disulfide linkage to fluorescein was conducted as follows: Fluorescein isothiocyanate (FITC) was used as the fluorescein source. Cystamine dihydrochloride (0.180 g, 1.18 mmol) was dissolved in 5 mL of methanol, 2 mL H₂O, and 40 μL of triethylamine. In a separate flask, FITC (0.051 g, 0.131 mmol) was dissolved in 5 mL methanol and 50 μL of triethylamine. The FITC solution was then added dropwise with stirring to the cystamine solution. The reaction was then maintained for 12 h at rt. Thereafter, the reaction volume was reduced to about 5 mL under vacuum. Cystamine-functionalized FITC was precipitated using a 10:1 solution of acetonitrile:methanol. The precipitate was washed three times with fresh 10:1 acetonitrile:methanol and dried in vacuum thereafter.

Oxidized carbon nanotubes (0.010 g, 0.832 mmol) were placed in a dry 50 mL round bottom flask. To the flask was added 10 mL of dry dimethylformamide (DMF), and the mixture was sonicated under nitrogen for 30 min to ensure complete carbon nanotube dispersion. To the dispersion was added 5,000 MW O-(2-Aminoethyl)-O′-methylpolyethylene glycol (PEG-NH₂) (0.083 g, 0.016 mmol), N,N′-dicyclohexylcarbodiimide (DCC, 0.172 g, 0.832 mmol) and a catalytic amount of dimethylaminopyridine (DMAP). The reaction was stirred under nitrogen at room temperature for 12 h to attach the poly(ethylene glycol) solubilizing groups. Some residual carboxylic acid groups remained unfunctionalized by the solubilizing groups. Following the attachment of the solubilizing groups, FITC-cystamine (0.045 g, 0.083 mmol) and additional DCC (0.172 g, 0.832 mmol) and DMAP were added. The reaction was stirred under nitrogen at rt for 12 h to react the FITC-cystamine with the residual carboxylic acid moieties. The solution was transferred to a 50,000 MWCO dialysis bag and dialyzed in standing DMF, with daily changing of the DMF solvent until no further fluorescence was detected in the DMF. The dialysis bag was then placed in running water dialysis for 5 d to isolate the oxidized carbon nanotube transporter component covalently bonded to the FITC fluorescent dye.

### Example 10

### Release of Disulfide-Bonded FITC from an Oxidized Carbon Nanotube Transporter Component in the Presence of a Thiol

The product of Example 9 (179 mg/L, 0.5 mL) was placed in a small beaker with a stirbar. To the solution was added 5 mL of water, to give a concentration of 16 mg/L. The FITC fluorescence of this solution at 525 nm (using an excitation wavelength of 488 nm) was 235 fluorescence units (arb. units). To the solution was then added dithiothreitol (DTT) (0.021 g, 0.133 mmol), and the solution was stirred for 1 h at rt. The reaction mixture was then filtered through a 0.22 μm Teflon membrane and the fluorescence at 525 nm (using an excitation wavelength of 488 nm) of the filtrate was 3,710 fluorescence units (arb. units). The marked increase in fluorescence is characteristic of the release of the FITC from the oxidized carbon nanotube transporter component. Upon release of the FITC from the oxidized carbon nanotube transporter component, the FITC's fluorescence was no longer quenched, and the marked increase in fluorescent intensity occurred. As shown by this example, reporter molecules may be selectively cleaved from the present compositions in the presence of a free thiol.

### Example 11

### Flow of an Oxidized Carbon Nanotube Transporter Component Through Lula Soil

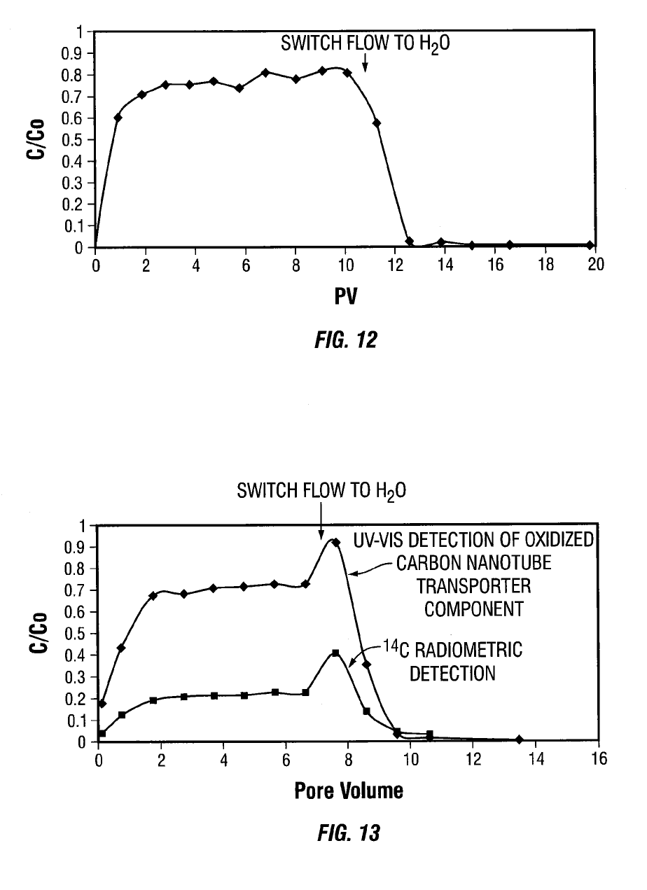

An oxidized carbon nanotube transporter component functionalized with poly(ethylene glycol) was synthesized as described in Examples 1 and 2. A water solution of the oxidized carbon nanotube transporter component (10 mg of carbon per liter) was eluted through a column packed with Lula soil. FIG. 12 shows an illustrative elution profile for an oxidized carbon nanotube transporter component eluted through Lula soil. The amount of the oxidized carbon nanotube transporter component that was able to pass through the column was determined by measuring the UV-VIS absorption eluted from the column as compared to the initial UV-VIS absorption at 763 nm (C/Cₒ). The amount of the solution eluted through the column was measured relative to the pore volume (PV) of the column. As shown in FIG. 12, approximately 80% of the oxidized carbon nanotube transporter component functionalized with PEG solubilizing groups passed through the column. Once the flow was switched over to pure water to flush the column, no further elution was detected. The lack of further elution indicates that the transporter component was not retained by the Lula soil.

To further investigate the elution behavior of the oxidized carbon nanotube transporter component through Lula soil, radiometric detection techniques were employed. FIG. 13 shows an illustrative elution profile from Lula soil for an oxidized carbon nanotube transporter component containing a sequestered ¹⁴C-radiolabelled aromatic compound. The oxidized carbon nanotube transporter component was synthesized analogously to that described in Example 8, except a desalting column was not used for purification. In other words, the oxidized carbon nanotube transporter component contained both sequestered radioactivity and unsequestered radioactivity. The amount of ¹⁴C aromatic compound in the eluent was measured radiometrically, and the amount of the oxidized carbon black transporter component alone was measured by UV-VIS spectroscopy. As shown in FIG. 13, radiometric analysis showed that only ˜20% of the radioactivity eluted through the column, while ˜70% of the oxidized carbon nanotube transporter component eluted. The eluted radioactivity was most likely due to radiolabelled compound remaining sequestered in the oxidized carbon nanotube transporter component, as the two traces have similar shapes. The remaining radioactivity likely remained bound to the Lula soil column, as a control experiment showed that when the ¹⁴C-radiolabelled aromatic compound was directly eluted through the Lula soil column with water, there was no detectable elution of radioactivity. If purification of the radiolabeled oxidized transporter component had been performed as described in Example 8, the unsequestered radioactivity would have been removed on the desalting column, and the percentage of eluted radioactivity would likely have been considerably higher from the Lula soil column.

### Example 12

### Breakthrough Characteristics of Various Poly(ethylene glycol)-Functionalized Transporter Components

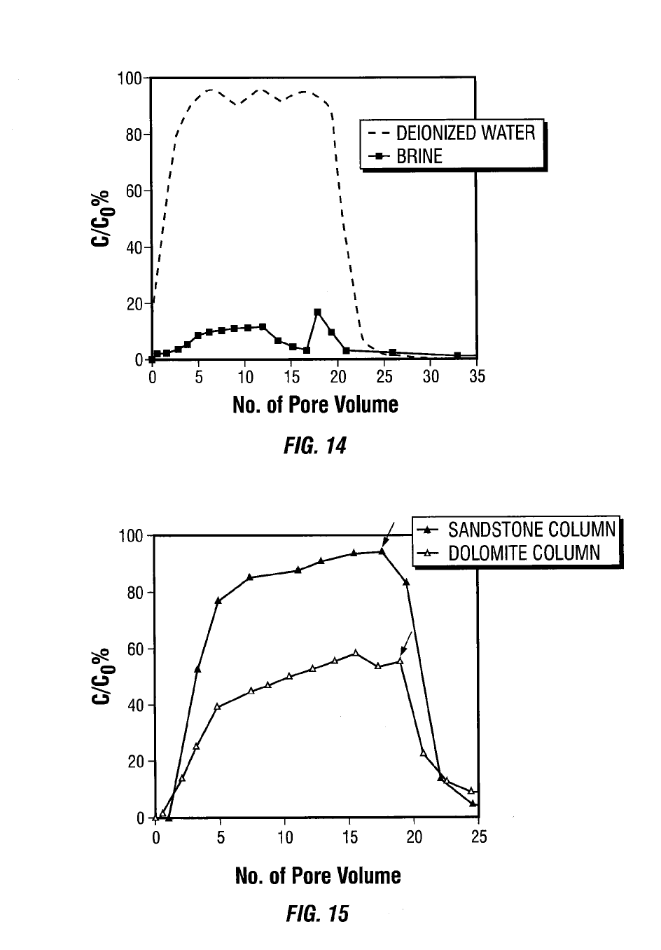

To better assess the ability of the various transporter components to move through representative porous soil media, breakthrough properties were thoroughly evaluated using soils (dolomite and sandstone) and simulated aqueous conditions commonly found in geological structures. FIG. 14 shows illustrative dolomite breakthrough plots for an oxidized graphene nanoribbon transporter component dissolved in deionized water and brine. For each breakthrough plot, the oxidized graphene nanoribbon transporter component was dissolved in either deionized water or brine and flowed through a column of dolomite at a flow rate of 8.31 mL/hr. The observed concentration of the eluent was reported as a fraction of the original concentration (C/C₀). As shown in FIG. 14, the breakthrough was approximately 95% when deionized water was used, meaning that only few of the oxidized graphene nanoribbons were retained on the column. However, when brine was used as the eluent, the breakthrough was less than 20%, meaning that a majority of the oxidized graphene nanoribbons were retained on the column. As will be shown hereinafter, transporter components retained on the column were removable by simply flushing the column with pure deionized water. Poly(ethylene glycol) functionalized graphene oxide exhibited similar breakthrough behavior to that of the oxidized graphene nanoribbons.

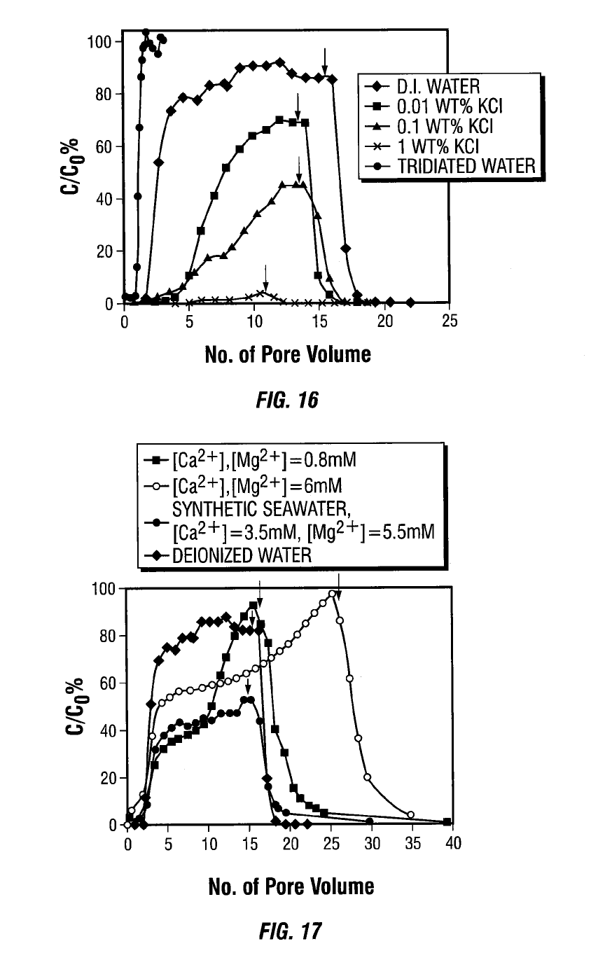

In contrast to oxidized graphene nanoribbon and oxidized graphene transporter components, oxidized carbon nanotube transporter components and oxidized carbon black transporter components exhibited much higher breakthroughs from dolomite. FIG. 15 shows illustrative sandstone and dolomite breakthrough plots for an oxidized carbon nanotube transporter component dissolved in brine. As shown in FIG. 15, the breakthrough was very high on sandstone (>85%) but considerably lower on dolomite (˜50%), although this value was significantly higher than that observed for oxidized graphene nanoribbon or oxidized graphene transporter components on dolomite under comparable conditions. Arrows in the breakthrough plots indicate the point at which pure deionized water was substituted for the transporter component solution in brine. The rapid drop in concentration indicates that the oxidized carbon nanotube transporter component was efficiently flushed from them column with deionized water in both cases. FIG. 16 shows illustrative dolomite breakthrough plots of oxidized carbon nanotube transporter components in the presence of various salt solution concentrations. Breakthrough of tritiated water is also presented for comparison in FIG. 16. As shown in FIG. 16, the breakthrough on dolomite steadily decreased as the salt concentration was increased. Possible reasons for the retention of the various transporter components on dolomite but not sandstone in the presence of aqueous salt solutions are considered in further detail hereinafter.

Without being bound by theory or mechanism, Applicants believe that with increasing ionic strength, the various transporter components deposit more readily on dolomite than on sandstone due to an increasing propensity to form a colloidal suspension. Like monovalent cations, divalent cations can also negatively impact the breakthrough of the transporter component. In addition to the aforesaid impact on ionic strength, divalent cations such as, for example, Mg²⁺ and Ca²⁺ can form salt bridges between the various transporter components via unfunctionalized carboxylic acid groups on the transporter component surface. Salt bridge formation is also capable of forming large aggregates of the transporter component, thereby resulting in deposition on dolomite surfaces and pores therein. Evidence that unfunctionalized carboxylic acid groups remain on the poly(ethylene glycol)-functionalized oxidized carbon nanotubes comes from this material's zeta potential of −35 mV.

Mg²⁺ and Ca²⁺ are commonly found in aqueous reservoirs associated with oilfields and on dolomite rock surfaces, and their impact on breakthrough has been considered separately from monovalent cations for this reason. FIG. 17 shows illustrative dolomite breakthrough plots of an oxidized carbon nanotube transporter component in the presence of various concentrations of divalent metal cations. As shown in FIG. 17, a similar concentration effect to that seen with monovalent metal cations was also noted with divalent metal cations. Generally, increased divalent metal cation concentration negatively impacted breakthrough. However, unlike the monovalent metal cations, the breakthrough plots exhibited an initial rapid rise before reaching a plateau value, and at extended elution volumes thereafter, a further increase in breakthrough was observed in the presence of divalent metal cations. In general, in the presence of divalent metal cations, a greater elution volume was required to reach the ultimate breakthrough plateau value. In FIG. 17, synthetic seawater is a mixture of salts in a water solution having the following composition: CaCl₂ (3.5 mM), MgCl₂ (5.5 mM), KCl (19.8 mM), NaCl (0.5 M), Na₂SO₄ (0.5 mM), and NaHCO₃ (2.0 mM).

Sandstone is also a major component of oil reservoirs. In contrast to dolomite, breakthrough in sandstone was uniformly high for the various transporter components in both water and aqueous salt solutions. Whereas dolomite is rich in Ca²⁺ and Mg²⁺, sandstone is predominantly silica and has a negatively charged surface at neutral pH. The breakthrough of aggregated transporter components did not appear to be adversely impacted on the negatively charged sandstone surface due to the lack of an efficient interaction with the surface. In contrast, relatively strong ion pairing with dolomite occurs in the presence of an aqueous salt solution. However, the ion pair can be easily disrupted by changing the ionic strength of the solution.

### Example 13

### Breakthrough Characteristics of Various Poly(vinyl alcohol)-Functionalized Transporter Components

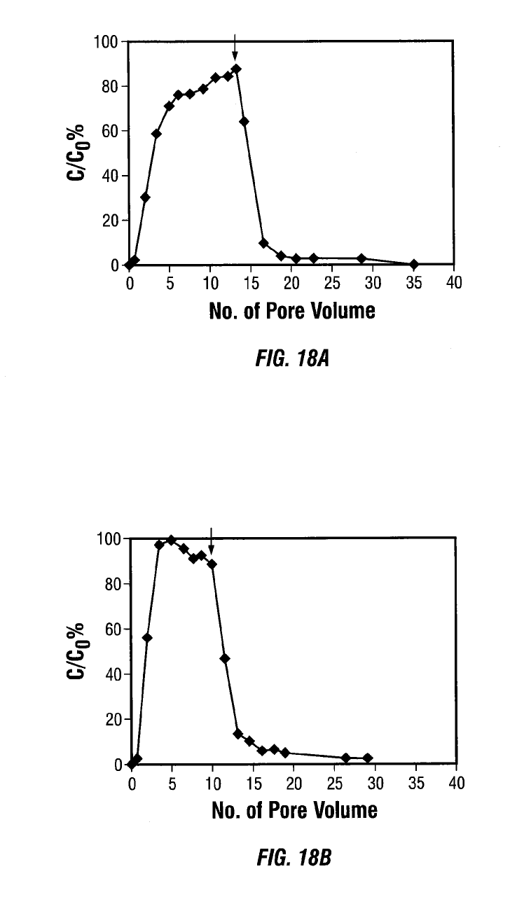

Efforts to improve the breakthrough behavior focused on replacement of the poly(ethylene glycol) solubilizing groups with poly(vinyl alcohol) solubilizing groups. Oxidized carbon nanotubes functionalized with poly(vinyl alcohol) solubilizing groups had a lower charge than did oxidized carbon nanotubes comparably functionalized with poly(ethylene glycol), as evidenced by their lower zeta potential of −20 mV. This result suggests that fewer carboxylic acids remained unfunctionalized upon poly(vinyl alcohol) functionalization. FIG. 18A shows an illustrative sandstone breakthrough plot for poly(vinyl alcohol)-functionalized oxidized carbon nanotubes dissolved in brine. FIG. 18B shows an illustrative dolomite breakthrough plot for poly(vinyl alcohol)-functionalized oxidized carbon nanotubes dissolved in brine. As shown in FIGS. 18A and 18B, the poly(vinyl alcohol)-functionalized oxidized carbon nanotubes both exhibited excellent breakthrough behavior, particularly on dolomite, where the breakthrough was significantly enhanced compared to that achieved with poly(ethylene glycol) solubilizing groups.

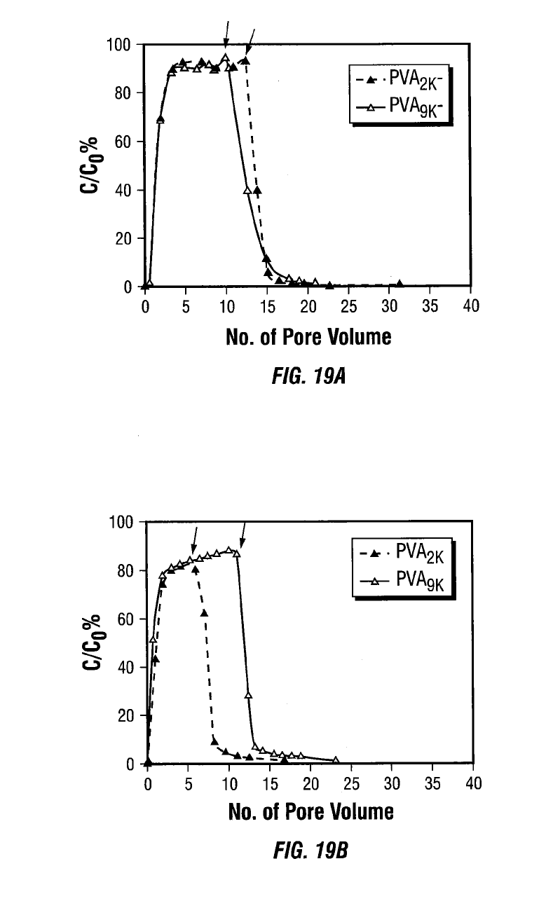

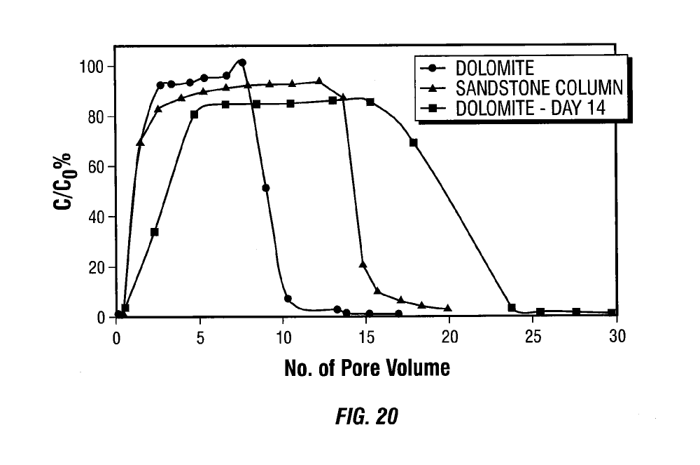

FIGS. 19A and 19B show illustrative sandstone (FIG. 19A) and dolomite (FIG. 19B) breakthrough plots in brine for oxidized carbon nanotube transporter components functionalized with various molecular weight poly(vinyl alcohol) solublizing groups (2,000 and 9,000 molecular weight). As shown in FIGS. 19A and 19B, there was very little apparent impact on the breakthrough behavior upon changing the poly(vinyl alcohol) molecular weight.

Due to the improved breakthrough properties observed with poly(vinyl alcohol) solubilizing groups, other transporter components functionalized with these solublizing groups were synthesized and tested. To this end, oxidized carbon black was synthesized and functionalized with 2,000 molecular weight poly(vinyl alcohol). FIG. 20 shows illustrative sandstone and dolomite breakthrough plots for poly(vinyl alcohol)-functionalized oxidized carbon black in brine. As shown in FIG. 20, the poly(vinyl alcohol)-functionalized oxidized carbon black exhibited excellent breakthrough behavior on both sandstone and dolomite. Comparative dolomite breakthrough behavior after the transporter component solution in brine had aged for 14 days showed little degradation in the breakthrough characteristics.

From the foregoing description, one of ordinary skill in the art can easily ascertain the essential characteristics of this disclosure, and without departing from the spirit and scope thereof, can make various changes and modifications to adapt the disclosure to various usages and conditions. The embodiments described hereinabove are meant to be illustrative only and should not be taken as limiting of the scope of the disclosure, which is defined in the following claims.

What is claimed is the following:

1. A composition comprising:
a transporter component comprising an amphiphilic nanomaterial and a plurality of solubilizing groups covalently bonded to the amphiphilic nanomaterial; and
a signaling component comprising a plurality of reporter molecules associated with the transporter component;
wherein at least a portion of the plurality of reporter molecules is releasable from the transporter component upon exposure to at least one analyte of interest.

2. The composition of claim 1, wherein the reporter molecules are all the same.

3. The composition of claim 1, wherein the reporter molecules comprise at least two different types of reporter molecules.

4. The composition of claim 1, wherein the transporter component is water soluble and the reporter molecules are not water soluble.

5. The composition of claim 1, wherein the reporter molecules are covalently bonded to the transporter component.

6. The composition of claim 1, wherein the reporter molecules are not covalently bonded to the transporter component.

7. The composition of claim 1, wherein a first portion of the reporter molecules is covalently bonded to the transporter component and a second portion of the reporter molecules is not covalently bonded to the transporter component.

8. The composition of claim 7, wherein the first portion and the second portion comprise different types of reporter molecules.

9. The composition of claim 7, wherein the first portion and the second portion are operable to detect different analytes of interest.

10. The composition of claim 1, wherein the reporter molecules are selected from the group consisting of fluorescent dyes, UV-active molecules, isotopically enriched molecules, radiolabeled molecules, metal nanoparticles and molecules that are sensitive to the presence of heavy metals.

11. The composition of claim 1, wherein the amphiphilic nanomaterial is selected from the group consisting of functionalized carbon nanotubes, graphene oxide, graphene oxide nanoribbons, oxidized carbon black particles, and metal nanoparticles;
wherein the carbon nanotubes comprise oxidized carbon nanotubes.

12. The composition of claim 1, wherein the amphiphilic nanomaterial comprises silica nanoparticles.

13. The composition of claim 1, wherein the solubilizing groups comprise water-soluble polymers.

14. The composition of claim 13, wherein the water-soluble polymers are selected from the group consisting of poly(ethylene glycol) (PEG), poly(propylene glycol) (PPG), poly(vinyl alcohol) (PVA), poly(ethylene imine) (PEI), poly(acrylic acid), poly(hydroxyalkyl ester), PLURONICS, saccharides, polysaccharides, carboxymethyl cellulose, and combinations thereof.

15. The composition of claim 1, wherein the at least one analyte of interest comprises petroleum.

16. The composition of claim 1, wherein the composition is operable to flow through a porous medium.

17. The composition of claim 16, wherein the porous medium is selected from the group consisting of soil, rock formations, and oil-containing geological formations.

18. The composition of claim 1, wherein the composition is stable in an aqueous salt solution.

19. The composition of claim 1, wherein the composition is responsive to at least one physical property of an aqueous environment;
wherein the at least one physical property is selected from the group consisting of presence or absence of an analyte of interest in the aqueous environment, relative abundance of water, pH, redox potential, electrolyte concentration, pressure, temperature, and combinations thereof.

20. A composition comprising:
a transporter component comprising an amphiphilic nanomaterial and a plurality of solubilizing groups covalently bonded to the amphiphilic nanomaterial; and
a signaling component comprising a first plurality of reporter molecules covalently bonded to the transporter component;
wherein at least a portion of the first plurality of reporter molecules is cleavable from the transporter component upon exposure to at least one analyte of interest.

21. The composition of claim 20, wherein the reporter molecules are covalently bonded to the transporter component by an ester bond.

22. The composition of claim 20, wherein the reporter molecules are covalently bonded to the transporter component by a disulfide bond.

23. The composition of claim 20, further comprising:
a second plurality of reporter molecules not covalently bonded to the transporter component;
wherein at least a portion of the second plurality of reporter molecules is releasable from the transporter component upon exposure to at least one analyte of interest; and
wherein the first plurality of reporter molecules and the second plurality of reporter molecules are operable to detect different analytes of interest.

24. The composition of claim 20, further comprising:
a second plurality of reporter molecules covalently bonded to the transporter component;
wherein at least a portion of the second plurality of reporter molecules is cleavable from the transporter component upon exposure to at least one analyte of interest; and
wherein the first plurality of reporter molecules and the second plurality of reporter molecules are operable to detect different analytes of interest.

25. A composition comprising:
a transporter component comprising an amphiphilic nanomaterial and a plurality of solubilizing groups covalently bonded to the amphiphilic nanomaterial;
wherein the transporter component is operable to become covalently bonded to a reporter molecule upon exposure to at least one analyte of interest.

26. The composition of claim 25, wherein the at least one analyte of interest comprises the reporter molecule.

27. The composition of claim 25, further comprising:
a plurality of reporter molecules non-covalently associated with the transporter component;
wherein at least a portion of the plurality of reporter molecules is operable to become covalently bonded to the transporter component upon exposure to the at least one analyte of interest.

28. A composition comprising:
a transporter component comprising an amphiphilic nanomaterial and a plurality of solubilizing groups covalently bonded to the amphiphilic nanomaterial;
wherein the amphiphilic nanomaterial is collapsible at a predetermined pressure.

29. The composition of claim 28, wherein the amphiphilic nanomaterial comprises a metal nanoparticle.

30. The composition of claim 29, wherein the metal nanoparticle is hollow.

31. The composition of claim 28, further comprising:
a signaling component comprising a plurality of reporter molecules associated with the transporter component;
wherein at least a portion of the plurality of reporter molecules is releasable from the transporter component upon exposure to at least one analyte of interest.

32. The composition of claim 31, wherein the plurality of reporter molecules are covalently bonded to the transporter component.

33. The composition of claim 31, wherein the plurality of reporter molecules are not covalently bonded to the transporter component.

34. A method comprising:
a) providing a composition comprising:
a transporter component comprising an amphiphilic nanomaterial and a plurality of solubilizing groups covalently bonded to the amphiphilic nanomaterial; and
a signaling component comprising a plurality of reporter molecules non-covalently associated with the transporter component;
wherein the plurality of reporter molecules are present in a first concentration in the composition;
b) exposing the composition to a liquid medium comprising at least one analyte of interest;
wherein at least a portion of the plurality of reporter molecules is released from the transporter component upon exposure to the at least one analyte of interest;
c) after exposing, recovering the composition from the liquid medium;
wherein the plurality of reporter molecules are present in a second concentration in the composition after exposing; and
d) assaying the composition to determine the second concentration.

35. The method of claim 34, wherein a ratio of the second concentration to the first concentration can be correlated with an amount of the at least one analyte of interest in the liquid medium.

36. The method of claim 34, wherein the liquid medium is selected from the group consisting of a geological formation, a wastewater source, a ground water source, and a surface water source.

37. The method of claim 34, further comprising:
e) assaying the liquid medium for the portion of the plurality of reporter molecules released from the transporter component.

38. A method comprising:
a) providing a composition comprising:
a transporter component comprising an amphiphilic nanomaterial and a plurality of solubilizing groups covalently bonded to the amphiphilic nanomaterial; and
a signaling component comprising a plurality of reporter molecules covalently bonded to the transporter component;
wherein the plurality of reporter molecules are present in a first concentration in the composition;
b) exposing the composition to a liquid medium comprising at least one analyte of interest;
wherein at least a portion of the plurality of reporter molecules is cleaved from the transporter component upon exposure to the at least one analyte of interest;
c) after exposing, recovering the composition from the liquid medium;
wherein the plurality of reporter molecules are present in a second concentration in the composition after exposing; and
d) assaying the composition to determine the second concentration.

39. The method of claim 38, wherein the reporter molecules are released from the transporter component upon being cleaved.

40. The method of claim 38, wherein a ratio of the second concentration to the first concentration can be correlated with an amount of the at least one analyte of interest in the liquid medium.

41. The composition of claim 38, wherein the liquid medium is selected from the group consisting of a geological formation, a wastewater source, a ground water source, and a surface water source.

42. The method of claim 38, wherein the reporter molecules are covalently bonded to the transporter component by an ester bond.

43. The method of claim 38, wherein the reporter molecules are covalently bonded to the transporter component by a disulfide bond.

44. The method of claim 43, wherein the at least one analyte of interest comprises a sulfur-containing compound.

45. The method of claim 38, further comprising:
e) assaying the liquid medium for the portion of the plurality of reporter molecules cleaved from the transporter component.

46. A method comprising:
a) providing a composition comprising:
a transporter component comprising an amphiphilic nanomaterial and a plurality of solubilizing groups covalently bonded to the amphiphilic nanomaterial;
b) exposing the composition to a liquid medium comprising at least one analyte of interest;
wherein at least a portion of the at least one analyte of interest becomes associated with the transporter component during exposing;
c) after exposing, recovering the composition from the liquid medium; and
d) assaying the composition to determine a concentration of the at least one analyte of interest in the composition.

47. The method of claim 46, wherein the concentration of the at least one analyte of interest in the composition can be correlated with an amount of the at least one analyte of interest in the liquid medium.

48. The method of claim 46, wherein the liquid medium is selected from the group consisting of a geological formation, a wastewater source, a ground water source, and a surface water source.

49. A method comprising:
a) providing a composition comprising:
a transporter component comprising an amphiphilic nanomaterial and a plurality of solubilizing groups covalently bonded to the amphiphilic nanomaterial; and
a signaling component comprising a plurality of reporter molecules associated with the transporter component;
wherein the plurality of reporter molecules are present in a first concentration in the composition;
b) injecting the composition into a geological formation containing at least one analyte of interest;
wherein at least a portion of the plurality of reporter molecules is released from the transporter component upon exposure to the at least one analyte of interest;
c) recovering the composition from the geological formation after a period of time;
wherein the plurality of reporter molecules are present in a second concentration in the composition after being recovered; and
d) assaying the composition to determine the second concentration;
wherein a ratio of the second concentration to the first concentration can be correlated with an amount of the at least one analyte of interest in the geological formation.

50. The method of claim 49, wherein the composition is injected into the geological formation in a first location and recovered in a second location.

51. The method of claim 49, wherein the composition is injected into the geological formation and recovered from the geological formation in the same location.

52. The method of claim 49, further comprising:
e) assaying the geological formation for the portion of the plurality of reporter molecules released from the transporter component.

53. A method comprising:
a) providing a first composition comprising:
a transporter component comprising an amphiphilic nanomaterial and a plurality of solubilizing groups covalently bonded to the amphiphilic nanomaterial; and
a signaling component comprising a first identification tag covalently bonded to the transporter component;
b) injecting the first composition into a geological formation;
c) providing a second composition comprising:
a transporter component comprising an amphiphilic nanomaterial and a plurality of solubilizing groups covalently bonded to the amphiphilic nanomaterial; and
a signaling component comprising a second identification tag covalently bonded to the transporter component;
d) injecting the second composition into the geological formation;
wherein a first period of time separates the injection of the first composition and the injection of the second composition; and
e) assaying the geological formation for the presence of the first composition and the second composition.

54. The method of claim 53, wherein a concentration of the first composition in the geological formation and a concentration of the second composition in the geological formation are diagnostic of physical changes that occur in the geological formation over the first period of time.

55. The method of claim 53, wherein a concentration of the first composition in the geological formation and a concentration of the second composition in the geological formation are diagnostic of an internal structure of the geological formation.

56. The method of claim 53, wherein a time taken for the first composition to be detected and a time taken for the second composition to be detected can be correlated with a distance that the first composition and the second composition travelled in the geological formation.

57. The method of claim 53, wherein the signaling components of first composition and the second composition further comprise a plurality of reporter molecules associated with the transporter component.

58. The method of claim 53, wherein the first identification tag and the second identification tag are selected from the group consisting of fluorescent dyes, radiolabelled molecules and isotopically labeled molecules.

59. The method of claim 53, further comprising:
f) providing a third composition comprising:
a transporter component comprising an amphiphilic nanomaterial and a plurality of solubilizing groups covalently bonded to the amphiphilic nanomaterial; and
a signaling component comprising a third identification tag covalently bonded to the transporter component;
g) injecting the third composition into the geological formation;
wherein a second period of time separates the injection of the second composition and the injection of the third composition; and
h) assaying the geological formation for the presence of the first composition, the second composition and the third composition.

60. The method of claim 53, wherein at least one of the first identification tag and the second identification tag is cleavable from the transporter component.

61. A method comprising:
a) providing a composition comprising:
a transporter component comprising an amphiphilic nanomaterial and a plurality of solubilizing groups covalently bonded to the amphiphilic nanomaterial; and
a signaling component comprising a plurality of reporter molecules associated with the transporter component and an identification tag covalently bonded to the transporter component;
b) injecting the composition into a geological formation in a first location;
c) recovering the composition from the geological formation in a second location over a period of time; and
d) analyzing the composition recovered from the second location.

62. The method of claim 61, wherein a time between injecting and recovering the composition can be correlated with an internal structure of the geological formation.

63. The method of claim 61, wherein a change in a concentration of the plurality of reporter molecules can be correlated with a concentration of at least one analyte of interest within the geological formation.

64. The method of claim 61, wherein the identification tag is selected from the group consisting of fluorescent dyes, radiolabelled molecules and isotopically labeled molecules.
